(19) United States
(12) Patent Application Publication
(10) Pub. No.: US 2020/0297015 A1
(43) Pub. Date: Sep. 24, 2020

(54) TRYPTOPHAN DERIVATIVES AS SWEETENERS

(71) Applicant: CHROMOCELL CORPORATION, North Brunswick, NJ (US)

(72) Inventors: Deshou JIANG, Belle Mead, NJ (US); Purvi SHAH, Bridgewater, NJ (US); Dennis SAWCHUK, Fanwood, NJ (US)

(73) Assignee: CHROMOCELL CORPORATION, North Brunswick, NJ (US)

(21) Appl. No.: 16/895,378

(22) Filed: Jun. 8, 2020

Related U.S. Application Data
(63) Continuation of application No. PCT/US2018/064473, filed on Dec. 7, 2018.

(30) Foreign Application Priority Data
Dec. 8, 2017 (US) 62596667

(51) Int. Cl. A23L 27/30; A23L 33/105; C07D 209/20

(52) U.S. Cl. CPC A23L 27/31; C07D 209/20; A23L 33/105

(57) ABSTRACT

The present disclosure provides compounds of Formula (I) and edible compositions comprising compounds of Formula (I) which are suitable for use as sweeteners, for example, by incorporation into edible compositions. Also provided herein are methods of preparing comestible compositions comprising compounds of Formula (I).

This application claims the benefit of U.S. provisional application No. 62/596,667 filed Dec. 8, 2017 which is incorporated by reference herein in its entirety.

### 1 FIELD

Provided herein are compounds of Formula (I) for use as non-caloric or low-caloric sweeteners, comestible compositions comprising such compounds, and methods of making such compounds and comestible compositions.

### 2 BACKGROUND

Natural sugars, such as sucrose, fructose and glucose, are utilized to provide a pleasant taste to beverages, foods, pharmaceuticals, oral hygienic products, and cosmetic products. However, such sugars are deleteriously caloric. Sucrose, the most widely used natural sweetener, provides superior sweetness characteristics, however its caloric content is a disadvantage, particularly in view of increasing global dietary health concerns associated with increased sucrose intake. Examples of such dietary health concerns include obesity, diabetes, cardiovascular disease, and tooth decay.

In view of such dietary health concerns, recommendations were made to reduce the amount of sugar in the diet which has paved the way for the introduction of non-caloric or low-caloric sweeteners into the market. Non-caloric or low-caloric sweeteners have played an increasing role in maintaining a healthy life-style by aiding in weight control and oral health. However, these sweeteners differ in taste from natural caloric sugars in ways that aggravate consumers. For example, non-caloric or low-caloric sweeteners exhibit a temporal profile, maximal response, flavor profile, mouth feel, and/or adaptation behavior that differ from natural caloric sugars, such as sucrose. Specifically, non-caloric or low-caloric sweeteners often exhibit delayed sweetness onset, lingering sweet aftertaste, bitter aftertaste, metallic taste, astringent taste, cooling taste and/or licorice-like taste. Saccharin, one of die most widely used non-caloric or low-caloric sweeteners, is often characterized by consumers as having a bitter and/or metallic aftertaste. Other high intensity sweeteners, such as sucralose and aspartame, have also been reported to have sweetness delivery problems, such as delayed onset and lingering of sweetness. See Wiet, S. G., et al. (1993). Fat concentration affects sweetness and sensory profiles of sucrose, sucralose, and aspartame. Journal of Food Science, 58(3), 599-602.

Further, many non-caloric or low-caloric sweeteners are synthetically derived which may be an undesirable characteristic for consumers seeking a natural sweetener alternative to sucrose. Thus, consumer desire and demand for natural non-caloric or low-caloric sweeteners that mimic the taste and quality of natural sweeteners such as sucrose remains high.

Derivatives of the amino acid tryptophan have been explored as potential non-caloric or low-caloric sweeteners. See, e.g., U.S. Pat. No. 3,899,592; Hengartner, U. et al., J. Org. Chem. 1979; 44(22):3741-3747; U.S. Pat. No. 4,316,847; Fukuda, J. et al., Appl. Microbiol. 1971; 21(5):841-3; each of which is incorporated herein by reference its entirety. In particular, 6-chloro-D-tryptophan has been identified as being 1000 times sweeter than sucrose. See Optimising Sweet Taste in Foods, 213 (W. J. Spillane. eds., 2006). However, none of these derivatives have been accepted by the FDA for use as food additives in the United States.

Accordingly, there is a strong desire to introduce new non-caloric or low-caloric sweetening compounds having improved taste mid delivery characteristics to consumers, as well as compositions containing such compounds. In addition, there is a need to introduce new food and beverage products incorporating these non-caloric or low-caloric sweetening compounds with such desirable characteristics.

### 1. SUMMARY

The present disclosure provides compounds that are useful as sweeteners, edible compositions comprising such compounds, and methods of preparing such edible compositions. The present disclosure further provides a method of providing sweet taste to an edible composition, such as a food, consumer or pharmaceutical product, in a subject. The present disclosure also provides a method of modulating, particularly enhancing or potentiating the activation of a sweet taste receptor.

Other aspects of the present disclosure include edible compositions, such as beverage compositions, concentrates (for use in, e.g., beverage compositions), food products, pharmaceutical preparations and table-top sweeteners comprising the compositions of the present disclosure; methods for preparing an edible composition; methods for reducing caloric intake; methods of enhancing activation of a sweet taste receptor and methods of synthesizing the sweet taste modulators of the present disclosure. Particular embodiments of the disclosure are set forth in the following numbered paragraphs:
1. A comestible composition comprising at least one compound of Formula (I):

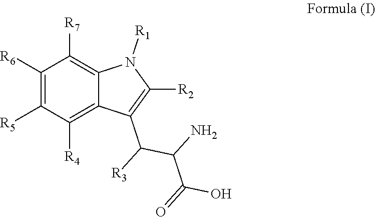

or a comestibly or biologically acceptable salt or stereoisomeric form thereof, wherein:
R₁ is hydrogen or C1-C6 alkyl;
R₂-R₅ and R₇ are each independently hydrogen, halogen, C1-C6 alkyl, C1-C6 alkoxy, hydroxyl or trifluoromethyl; and
R₆ is I, hydrogen, C4-C6 alkyl, C4-C6 alkoxy or hydroxyl.
   2. The comestible composition of claim 1, wherein R₁, R₂, R₃, R₅, R₆, and R₇ are independently at each occurrence hydrogen.
   3. The comestible composition of claim 2, wherein R₄ is methyl.
   4. The comestible composition of any one of claims 1-3, wherein the at least one compound is 2-amino-3-(4-methyl-1H-indol-3-yl)propanoic acid.
   5. The comestible composition of claim 1, wherein R₁, R₂, R₃, R₄, R₆ and R₇ are independently at each occurrence hydrogen.
   6. The comestible composition of claim 5, wherein R₅ is methyl.
   7. The comestible composition of any one of claims 1 or 5-6, wherein the at least one compound is 2-amino-3-(5-methyl-1H-indol-3-yl)propanoic acid.
   8. The comestible composition of claim 1, wherein R₁, R₂, R₃, R₅, R₆, and R₇ are independently at each occurrence hydrogen.
   9. The comestible composition of claim 8wherein R₂ methyl.
   10. The comestible composition of any one of claim 1 or 8-9, wherein the at least one compound is 2-amino-3-(2-methyl-1H-indol-3-yl)propanoic acid.
   11. The comestible composition of any one of claims 1-10, wherein the comestible composition comprises racemic mixture of the at least one compound.
   12. The comestible composition of any one of claims 1-10, wherein the at least one compound is the D-isomer.
   13. The comestible composition of any one of claims 1-10, wherein the at least one compound is the L-isomer.
   14, The comestible composition of any one of claims 1-13, wherein the comestibly biologically acceptable salt is selected from the group consisting of a sodium salt, a potassium salt, or a calcium salt.
   15. The. comestible composition of any one of claims 1-14, wherein the comestible or at most 95% by weight of the compound of Formula (I).
   16, The comestible composition of any one of claims 1-14, wherein the comestible composition comprises at most 0.1%, 0.5%, 1%, 2.5%, 5%, 7.5%, 10%, 25%, 50%, 75%, 90%, composition comprises at least 0.5%, 1%, 2.5% 5%, 7.5%, 10%, 25%, 50%, 75%, 90%, or at least 95% by weight of the compound of Formula (I),
   17. The comestible composition of any one of claims 1-16, wherein the comestible composition is enclosed in a package for storing and dispensing the comestible composition.
   18. The comestible composition of any one of claims 1-17, wherein the at least one compound has an EC₅₀ value of at most 0.01 mM, 0.05 mM, 0.1 mM, 5 mM, 10 mM, 20 mM, or at most 30 mM in a cell-based sweet receptor assay, wherein the cell-based assay comprises contacting the at least one compound with a cell expressing TAS1R2+TAS1R3.
   19. The comestible composition of claim 18, wherein the cell-based assay further comprises measuring intracellular Ca²⁺ concentration using a Ca²⁺-sensitive fluorescent dye.
   20. The comestible composition of claim 18, wherein the EC₅₀ value is calculated by fitting the data from the cell-based assay to the Hill's equation.
   21. The comestible composition of any one of claims 1-20, wherein the at least one compound imparts a more sugar-like characteristic to the comestible composition than a comestible composition without the at least one compound.
   22. The comestible composition of claim 21, wherein the sugar-like characteristic is selected from the group consisting of maximal response, flavor profile, temporal profile, adaptation behavior, mouthfeel, concentration/response function behavior, tastant and flavor/sweet taste interactions, and temperature effects.
   23. The comestible composition of any one of claims 1-22, wherein the at least one compound is present in an aqueous solution in an amount sufficient to impart a maximum sweetness intensity equivalent to that of a 20% aqueous solution of sucrose by weight.
   24. The comestible composition of any one of claims 1-23, wherein 50 mg-100 mg of the at least one compound in 100 mg of water is equivalent to 1 degree Brix.
   25. The comestible composition of any one of claims 1-23, wherein the comestible composition provides from about 1 to about 12 degrees Brix when added to an unsweetened beverage.
   26. The comestible composition of any one of claims 1-25, wherein the at least one compound suppresses, reduces, or eliminates at least one undesirable taste.
   27. The comestible composition of claim 26, wherein the at least one undesirable taste is selected from the group consisting of delayed sweetness onset, lingering sweet aftertaste, metallic taste, bitter taste, cooling sensation taste, menthol-like taste, or licorice-like taste.
   28. The comestible composition of any one of claims 1-27, wherein the at least one compound is separated from its naturally occurring environment if it is derived from a natural source or product.
   29. The comestible composition of any one of claims 1-28, wherein the at least one compound is synthesized.
   30. The comestible composition of any one of claims 1-29, wherein the at least one compound is bio-synthesized.
   31. The comestible composition of any one of claims 1-30, wherein the at least one compound is isolated and purified.
   32. A comestible composition comprising a compound having the following structure:

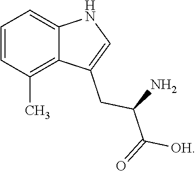 [SMILES: Cc1cccc2[nH]cc(C[C@@H](N)C(=O)O)c12]

33. A comestible composition comprising a compound having the following structure:

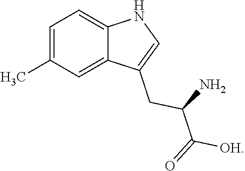 [SMILES: Cc1ccc2[nH]cc(C[C@@H](N)C(=O)O)c2c1]

34. A comestible composition comprising a compound having the following structure:

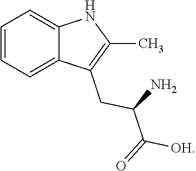 [SMILES: Cc1[nH]c2ccccc2c1C[C@@H](N)C(=O)O]

35. The comestible composition of any one of claims 32-34, wherein the compound has enhanced solubility over other isomers or racemic mixtures of the compound.
36. A dietary sweetener composition comprising the comestible composition of any one of claims 1-34.
37. The dietary sweetener composition of claim 35 further comprising at least one additive in addition to the compound of Formula (I).
38. The dietary sweetener composition of claim 37, wherein the additive is selected from the group consisting of carbohydrates, polyols, amino acids and their corresponding salts, poly-amino acids and their corresponding salts, sugar acids and their corresponding salts, nucleotides, organic acids, inorganic acids, organic salts including organic acid salts and organic base salts, inorganic salts, bitter compounds, flavorants and flavoring ingredients, astringent compounds, proteins or protein hydrolysates, surfactants, emulsifiers, weighing agents, gums, antioxidants, colorants, flavonoids, alcohols, polymers and combinations thereof.
39. The dietary sweetener composition of any one of claims 35-38, wherein the sweetener composition is a tabletop sweetener composition.
40. The dietary sweetener composition of any one of claims 35-38, wherein the sweetener composition is a zero-, low-, or mid-calorie sweetener composition, optionally containing caffeine.
41. A consumable comprising the comestible composition of any one of claims 1-34 or the dietary sweetener composition of any one of claims 35-40.
42. The consumable of claim 41, wherein the consumable is a beverage or a fowl.
43. The consumable of any one of claims 41 or 42, wherein the consumable is a beverage.
44. The consumable of claim 43, wherein the beverage is selected from a zero-, low-, and mid-calorie beverage, optionally containing caffeine.
45. A beverage mix comprising the comestible composition of any one of claims 1-34 or the dietary sweetener composition of any one of claims 35-40.
46. The beverage mix of claim 45, wherein the mix is dry.
47. The beverage mix of claim 45, wherein the mix is a liquid concentrate.
48. A pharmaceutical composition comprising the comestible composition of any one of claims 1-34 or the dietary sweetener composition of any one of claims 35-40.
49. The pharmaceutical composition of claim 48, wherein the pharmaceutical composition further comprises an active pharmaceutical ingredient.
50. The pharmaceutical composition of any one of claims 48 or 49, wherein the pharmaceutical composition is formulated for oral administration, buccal administration, or sublingual administration.
51. The pharmaceutical composition of claim 50, wherein the comestible composition is applied as a coating to the pharmaceutical composition.
52. A method of preparing a comestible composition, comprising:
   a providing a comestibly acceptable carrier; and
   b. adding to the comestibly acceptable carrier at least one compound of Formula (I)

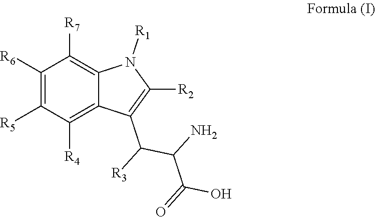

or a comestibly or biologically acceptable salt or stereoisomeric form thereof, wherein:
R1 is hydrogen or C1-C6 alkyl;
R₂-R₅ and R₇ are each independently hydrogen, halogen, C1-C6 alkyl, C1-C6 alkoxy, hydroxyl or trifluoromethyl; and
R₆ is I, hydrogen, C4-C6 alkyl, C4-C6 alkoxy or hydroxyl.
   53. The method of claim 52, wherein the at least one compound is 2-amino-3-(4-methyl-1H-indol-3-yl)propanoic acid.
   54. The method of claim 52, wherein the at least one compound is 2-amino-3-(5-methyl-1H-indol-3-yl)propanoic acid.
   55. The method of claim 52, wherein the at least one compound is 2-amino-3-(2-methyl-1H-indol-3-yl)propanoic acid.
   56. The method of any one of claims 52-54, wherein the at least one compound is separated from its naturally occurring environment if it is derived from a natural source or product.
   57. The method of any one of claims 52-56, wherein the at least one compound is synthesized.
   58. The method of any one of claims 52-57, wherein the at least one compound is bio-synthesized.
   59. The method of any one of claims 52-58, wherein the at least one compound is isolated and purified.
   60. A method for enhancing the sweetness of a consumable, comprising:
      a providing a consumable;
      b. adding to the consumable the comestible composition of any one of claims 1-34 or the dietary sweetener composition of any one of claims 35-40.
   61. A method for enhancing the sugar-like characteristics of a consumable, comprising:
      a providing a consumable;
      b. adding to the consumable the comestible composition of any one of claims 1-34 or the dietary sweetener composition of any one of claims 35-40.

### 2. BRIEF DESCRIPTION OF THE DRAWINGS

FIG. 1 shows the scheme for synthesizing 5-methyl D-tryptophan.

FIG. 2 shows the cell activity results for Compound 5.

FIG. 3 shows the cell activity results for Compound 8.

### 3. DETAILED DESCRIPTION

Provided herein are certain compounds of Formula (I) and comestible compositions comprising such compounds of Formula (I). Specific compounds of Formula (I) are provided in Section 3.2. Methods of synthesizing compounds of Formula (I) are provided in Section 3.3. Comestible compositions comprising compounds of Formula (I) are provided in Section 3.4. Methods of use are provided in Section 3.5.

### 3.1 Definitions

Chemistry terms used herein are used according to conventional usage in the art, as exemplified by “The McGraw-Hill Dictionary of Chemical Terms”, Parker S., Ed., McGraw-Hill, San Francisco, Calif. (1985).

As used herein, and unless otherwise specified, the terms “about” and “approximately,” when used in connection with doses, amounts, or weight percent of ingredients of a composition or a dosage form, mean a dose, amount, or weight percent that is recognized by one of ordinary skill in the art to provide a pharmacological effect equivalent to that obtained from the specified dose, amount, or weight percent . In certain embodiments, the terms “about” and “approximately,” when used in this context, contemplate a dose, amount, or weight percent within 30%. within 20%, within 15%, within 10%, or within 5%, of the specified dose, amount, or weight percent.

As used herein any numerical range recited herein includes all values from lower to upper value. For example, if a concentration range is stated as 10 ppm to 50 ppm it is intended that such values as 20 ppm to 40 ppm, 20 ppm to 30 ppm, or 10 ppm to 15 ppm, etc. are expressly enumerated in this specification. These are only examples of what is specifically intended, and all possible combinations of numerical values between and including the lowest value and the highest value enumerated are to be considered to be expressly stated in this application.

As used herein, and unless otherwise indicated, the term “active pharmaceutical ingredient” refers to any drug, drug formulation, medication, prophylactic agent, therapeutic agent, compound or other substance having biological activity. Suitable active pharmaceutical ingredients for the embodiments provided herein include, but are not limited to, medications for the gastrointestinal tract or digestive system, for the cardiovascular system, for the central nervous system, for pain or consciousness, for musculo-skeletal disorders, for the eye, for the ear, nose and oropharynx, for the respiratory system, for endocrine problems, for the reproductive system or urinary system, for contraception, for obstetrics and gynecology, for the akin, for infections and infestations, for immunology, for allergic disorders, for nutrition, for neoplastic disorders, for diagnostics, for euthanasia, or other biological functions or disorders.

As used herein, and unless otherwise indicated, the term “alkoxy” means an —OR radical or group, where R is alkyl as defined above, e.g., methoxy, ethoxy, propoxy, or 2-propoxy, n-, iso-, or tert-butoxy, and the like. In certain embodiments, preferred alkoxy groups of the invention have 1 to 6 carbon atoms. In other embodiments, preferred alkoxy groups of the invention have three or more carbon atoms, preferably 4 to 6 carbon atoms. An alkoxy group may be optionally substituted where allowed by available valences. Examples of substituted alkoxy groups include trifluoromethoxy, hydroxy methyl, hydroxyethyl, hydroxypropyl, and alkoxyalky) groups such as methoxymethyl, methoxyethyl, polyoxyethylene, polyoxopropylene, and similar groups. Unless specifically stated as “unsubstituted,” references to chemical moieties herein are understood to include substituted variants.

As used herein, unless otherwise specified, the term “alkyl” means a saturated straight chain or branched hydrocarbon chains having, for example, 1 to 20 carbon atoms. In some embodiments, the alkyl groups comprise “C1 to C6 alkyl” groups (alternatively termed “lower alkyl” groups) that include methyl, ethyl, propyl, iso-propyl n-butyl, iso-butyl, sec-butyl, t-butyl, pentyl, n-pentyl, tert-pentyl, neo-pentyl, iso-pentyl, 2-methylpentyl, 3-methylpentyl, 4-methylpentyl, 2,3-dimethylbutyl, hexyl, n-hexyl, tert-hexyl, neo-hexyl, iso-hexyl, sec-hexyl, and the like. In certain embodiments, preferred alkyl groups of the invention have 1 to 6 carbon atoms. In certain embodiments, preferred alkyl groups of the invention have three or more carbon atoms, preferably 4 to 6 carbon atoms. An alkyl group may be optionally substituted where allowed by available valences. Unless specifically stated as “unsubstituted,” references to chemical moieties herein are understood to include substituted variants.

As used herein, unless otherwise specified, the term “bitter tastant,” “bitter ligand,” or “bitter compound” refers to a compound that activates or that can be detected by a bitter taste receptor and/or confers the perception of a bitter taste in a subject. A bitter tastant also refers to a number of compounds that combine to activate or be detected by a bitter taste receptor and/or confer the perception of a bitter taste in a subject. A bitter tastant further refers to a compound that is enzymatically modified upon ingestion by a subject to activate or be detected by a bitter taste receptor and/or confer the perception of a bitter taste in a subject. Because the perception of bitter taste may vary from individual to individual, some individuals may describe a bitter tastant as a compound which confers a different kind of bitter taste compared to the kind of bitter taste perceived for the same compound by other individuals. The term bitter tastant also refers to a compound which confers a bitter taste. Those of skill in the art can readily identify and understand what is meant by a bitter tastant. Non-limiting examples of bitter tastants or substances including foods that comprise a bitter tastant and taste bitter include coffee, unsweetened cocoa, marmalade, bitter melon, beer, bitters, citrus peel, dandelion greens, escarole, quinine, magnesium salts, calcium salts, potassium salts, KCl, potassium lactate, acesulfame K, saccharin, rebaudioside A, rebaudioside C, stevioside, sucralose, tea polyphenols, brussel sprouts, asparagus, bitter gourd, wild cucumber, celery, hops, kohlrabi, radish leaf, ginseng, pumpkin, collard greens, kale, sparteine, caffeine, atropine, nicotine, urea, and strychnine. Further examples of bitter tastants include pharmaceuticals. Non-limiting examples of pharmaceuticals as bitter tastants include acetaminophen, ampicillin, azithromycin, chlorpheniramine, cimetidine, dextromethorphan, diphenhydramine, erythromycin, esomeprazole, guaifenesin, ibuprofen, penicillin, phenylbutazone, pseudoephedrine, ranitidine, sildenafil, spironolactone, and theophylline.

As used herein, and unless otherwise indicated, the terms “combination” or “combinations” refer to a mixture of two or more compounds of the invention. Combinations can include, but arc not limited to, a combination of one or more compounds of Formula (I), or comestibly or biologically acceptable salts, derivatives, diastereomers, or enantiomers thereof.

As used herein, and unless otherwise indicated, the terms “comestibly acceptable carrier” is a solid or liquid medium and/or composition that is used to prepare a comestible composition with the desired amount of a Compound provided herein in order to administer a compound provided herein (such as a compound of Formula (I), Compound 1, Compound 2, Compound 3, Compound 4, Compound 5, Compound 6, Compound 7, Compound 8, Compound 9, or a combination of any of the foregoing compounds) in a dispersed/diluted form.

As used herein, and unless otherwise indicated, the term “comestibly or biologically acceptable salt” refers to any comestibly or biologically acceptable salt, ester, or salt of such ester, of a compound of the present invention, which, upon ingestion, is capable of providing (directly or indirectly) a compound of the present invention, or a metabolite, residue or portion thereof, characterized by the ability to reduce the perception of a bitter taste attributed to a bitter tastant. In certain embodiments, preferred comestibly or biologically acceptable salts of a compound of Formula (I) arc hydrochloride salts.

As used herein, and unless otherwise indicated, the term “composition” is intended to encompass a product comprising the specified ingredient(s) (and in the specified amount(s), if indicated), as well as any product which results, directly or indirectly, from combination of the specified ingredient(s) in the specified amount(s).

As used herein, and unless otherwise indicated, the term “consumable” refers to a product suitable for oral use, such as eating or drinking. Therefore, a consumable is an edible compound or composition.

As used herein, and unless otherwise indicated, the term “effective amount” of a compound provided herein (such as a compound of Formula (I). Compound 1, Compound 2, Compound 3, Compound 4, Compound 5Compound 6, Compound 7, Compound 8 or Compound 9) meant a sufficient amount of one or more compounds in a composition that is sufficient to provide the desired regulation of a desired biological function, such as gene expression, protein function, or more particularly the induction of sweet taste perception in an animal or a human. As will be pointed out below, the exact amount required will vary from subject to subject and from composition to composition, depending on the species, age, general condition of the subject, specific identity and formulation of the comestible composition, etc. Thus, it is not possible to specify an exact “effective amount.” However, an appropriate effective amount can be determined one of ordinary skill in the art using only routine experimentation.

As used herein, and unless otherwise indicated, the term “EC₅₀” (i.e., half maximal effective concentration) refers to the molar concentration of a modulator which produces 50% of the maximum .possible effective response from that modulator.

As used herein, and unless otherwise indicated, the terms “enantiomerically pure” means a stereomerically pure composition of a compound having at least one chiral center.

As used herein, and unless otherwise indicated, the terms “excipient” refers to any inactive substance used as a vehicle for an active pharmaceutical ingredient, such as any material to facilitate handling, stability, dispersibility wettability, and/or release kinetics of an active pharmaceutical ingredient.

As used herein and unless otherwise indicated, the terms “halo” and “halogen” refer to the fluoro, chloro, bromo or iodo atoms. There can be one or more halogens, which are the same or different.

As used herein, and unless otherwise indicated, the term “hydrogen” means —H.

As used herein, and unless otherwise indicated, the term “hydroxy” means —OH,

As used herein, and unless otherwise specified, the term “low-calorie” refers to a dietary sweetener composition that has 75% or less of the calories that would be associated with a full calorie sweetener composition.

As used herein, and unless otherwise specified, the terms “mid-calorie” refers to a dietary sweetener composition that has a 50% reduction in calories that would be associated with a full calorie sweetener composition.

As used herein, and unless otherwise specified, the term “mix” refers to a product that is typically mixed by a consumer with an aqueous liquid or diluent (i.e., water, milk or other aqueous medium) to provide a ready-to-serve food or beverage. The mix may be in either in a powder, dry mix, concentrate, crystalline, spray dried or emulsion form. In some embodiments, the mix is relatively soluble in water, particularly hot water.

As used herein, and unless otherwise indicated, the term “modulator” refers to a compound or substance that alters the structure, conformation, biochemical or biophysical properties or functionality of a sweet taste receptor, either positively or negatively. The modulator can be a sweet taste receptor agonist (potentiator or activator) or antagonist (inhibitor or blocker), including partial agonists or antagonists, selective agonists or antagonists and inverse agonists, and can be an allosteric modulator. A substance or compound is a modulator even if its modulating activity changes under different conditions or concentrations or with respect to different forms of sweet taste receptors, e.g., naturally occurring form vs. mutant form, and different naturally-occurring allelic variants of a sweet taste receptor (e.g., due to polymorphism). As used herein, a modulator may affect the activity of a sweet taste receptor, the response of a sweet taste receptor to another regulatory compound or the selectivity of a sweet taste receptor. A modulator may also change die ability of another modulator to affect the function of a sweet taste receptor. A modulator may act upon all or upon a specific subset of sweet taste receptors. Modulators include, but are not limited to, potentiators, activators, inhibitors, agonists, antagonists and blockers.

As used herein, and unless otherwise indicated, the term “natural high-potency sweetener” refers to any sweetener found in nature which may be in raw, extracted, purified, or any other form, singularly or in combination thereof and characteristically have a sweetness potency greater than sucrose, fructose, or glucose, yet have less calories. Non-limiting examples of natural high-potency sweeteners suitable for embodiments provided herein include rebaudioside A, rebaudioside B, rebaudioside C (dulcoside B), rebaudioside D, rebaudioside E, rebaudioside F, rebaudioside M, rebaudioside N, Rebaudioside X, dulcoside A, rubusoside, stevia, stevioside, mogroside IV, mogroside V, Luo Flan Guo sweetener, siamenoside, monatin and its salts (monatin SS. RR, RS, SR), curculin, glycyrrhizic acid and its salts, thaumatin, monellin, mabinlin, brazzein, hemandulcin, phyllodulcin, glycyphyllin, phloridzin, trilobtain, baiyunoside, osladin, polypodoside A, pterocaryoside A, pterocaryoside B, mukurozioside, phlomisoside I, periandrin I, abrusoside A, and eyclocarioside I. Natural high-potency sweeteners also include modified natural high-potency sweeteners.

As used herein, and unless otherwise indicated, the terms “pharmaceutically acceptable salt” refers to a salt prepared from a pharmaceutically acceptable non-toxic acid or base including an inorganic acid and base and an organic acid and base. Suitable pharmaceutically acceptable base addition salts of a compound provided herein (such as a compound of Formula (I), Compound 1, Compound 2, Compound 3, Compound 4, Compound 5, Compound 6, Compound 7, Compound 8 or Compound 9) include, but are not limited to metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucaminc). and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromie, hydrochloric, isethionic, lactic, maleic, malic, mandelic, methanesuifonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromie, phosphoric, sulfuric, and methanesuifonic acids. Others are well known in the art, see for example, Remington's Pharmaceutical Sciences. 18th eds., Mack Publishing, Easton Pa. (1990) or Remington: The Science and Practice of Pharmacy, 19th eds., Mack Publishing, Easton Pa. (1995).

As used herein, and unless otherwise indicated, the term “stereoisomer” or “stereoisomeric form” refers to one stereoisomer of a compound of Formula (I) that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. A compound of Formula (I) can have chiral centers and can occur as racemates, individual enantiomers or diastereomers, and mixtures thereof. All such isomeric forms are included within the embodiments disclosed herein, including mixtures thereof. The use of stereomerically pure forms of such compounds, as well as the use of mixtures of those forms, are encompassed by the embodiments disclosed herein. For example, mixtures comprising equal or unequal amounts of the enantiomers of a particular compound of Formula (I) may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley Interscience, New York, 1981); Wilen. S. H., et al., Tetrahedron 33:2725 (1977); Eliel, E. L., Stereochemistry of Carbon Compounds (McGraw Hill, NY, 1962); and Wilen, S. H., Tables of Resolving Agents and Optical Resolutions p. 268 (E. L. Eliel, Ed., Univ. of Notre Dame Press, Notre Dame, Ind., 1972).

As used herein, unless otherwise specified, the term “subject” refers to a mammal. In preferred embodiments, the subject is human. In some embodiments, a subject is a domestic or laboratory animal, including but not limited to, household pets, such as dogs, cats, pigs, rabbits, rats. mice, gerbils, hamsters, guinea pigs, and ferrets. In some embodiments, a subject is a livestock animal. Non-limiting examples of livestock animals include: alpaca, bison, camel, cattle, deer, pigs, horses, llamas, mules, donkeys, sheep, goats, rabbits, reindeer, and yak. In one embodiment, the subject is a patient.

As used herein, and unless otherwise specified, a compound that is “substantially pure” is substantially free from other compounds (i.e., impurities). In certain embodiments, a compound that is substantially pure contains less than about 10%, 9%, 8%, 7%, 6%, 5%, 4%, 3%, 2%, 1%, 0.5%, 0.4%, 0.3%, 0.2%, 0.1%, 0.05%, or 0.01% of one or more other compounds on a weight basis. The detection of other compounds can be accomplished by any method apparent to a person of ordinary skill in the art, including, but not limited to, methods of chemical analysis, such as, e.g., mass spectrometry analysis, spectroscopic analysis, thermal analysis, elemental combustion analysis and/or chromatographic analysis.

As used herein, unless otherwise specified, the term “substituted” means a group may be substituted by one or more independent substituents, examples of which include, but are not limited to, halo, alkyl, alkoxy, trifluoromethyl, trifluoromethoxy, hydroxy, alkoxy, cycloalkyoxy, heterocylooxy, oxo, alkanoyl, alkylcarbonyl, cycloalkyl, aryl, aryloxy, aralkyl, alkanoyloxy, cyano, azido, amino, alkylamino, —S(O)₂OH, arylamino, aralkylamino, cycloalkylamino, heterocycloamino, mono and disubstituted amino in which the two substituents on the amino group are selected from alkyl, aryl, aralkyl, alkanoylamino, aroylamino, aralkanoylamino, substituted alkanoylamino, substituted arylamino, substituted aralkanoylamino, thiol, alkylthio, arylthio, aralkylthio, cycloalkylthio, heterocyclothio, alkylthiono, arylthiono, aralkylthiono, alkylsulfonyl, arylsulfonyl, aralkylsulfonyl, oxygen, sulfonamido (e.g., —SO₂NH₂), substituted sulfonamido, nitro, carboxy, carbamyl (e.g., —CONH₂), substituted carbamyl (e.g., —CONH alkyl, —CONH aryl, —CONH aralkyl or instances where there ere two substituents on the nitrogen selected from alkyl, aryl or aralkyl), alkoxycarbonyl, aryl, substituted aryl, guanidino and heterocyclo, such as indolyl, imidazolyl, furyl, thienyl, thiazolyl, pyrrolidyl, pyridyl, pyrimidyl, and the like.

As used herein, and unless otherwise indicated, the term “sugar” refers to a simple carbohydrate, such as a monosaccharide or a disaccliaride that delivers a primary taste sensation of sweetness. Non-limiting examples of sugar include glucose, fructose, galactose, sucrose, lactose, and maltose. In a preferred embodiment, sugar is sucrose.

As used herein, and unless otherwise indicated, the phrases “sugar-like characteristic,” “sugar-like taste,” “sugar-like sweet,” “sugary,” and “sugar-like” are synonymous. Sugar-like characteristics include any characteristic similar to that of sucrose and include, but are not limited to, maximal response, flavor profile, temporal profile, adaptation behavior, mouthfeel, concentration/response function behavior, tastant and flavor/sweet taste interactions, and temperature effects. These characteristics are dimensions in which the taste of sucrose is different from the tastes of natural and synthetic high-potency sweeteners. Whether or not a characteristic is more sugar-like is determined by expert taster or trained sensory panel assessments of sugar and compositions comprising at least one natural and/or synthetic high-potency sweetener, both with and without a sweet taste improving composition. Such assessments quantity similarities of the characteristics of compositions comprising at least one natural and/or synthetic high-potency sweetener, both with and without a sweet taste improving composition, with those comprising sugar. Suitable procedures for determining whether a composition has a more sugar-like taste are well known in the art.

As used herein, and unless otherwise modified, the term “suspension” refers to a system whereby very small particles (e.g., solid, semi-solid or liquid) are more or less uniformly dispersed in a different liquid or gaseous medium.

As used herein, and unless otherwise indicated, the term “sweet flavor” refers to the taste elicited by, for example, sugars. Non-limiting examples of compositions eliciting a sweet flavor include glucose, sucrose, fructose, saccharin, cyclamate, aspartame, acesulfame potassium, sucralose, alitame, and neotame. The amount of sweet flavor or the sweetness of a composition can be determined by, e.g., taste testing.

As used herein, and unless otherwise indicated, the term “synthetic high potency sweetener” refers to any composition which is not found naturally in nature and characteristically has a sweetness potency greater than sucrose, fructose, or glucose, yet have fewer or no calories. Non-limiting examples of synthetic high-potency sweeteners suitable for embodiments of this disclosure include sucralose, potassium acesulfame, aspartame, alitame, saccharin, neohesperidin dihydrochalcone, cyclamate, neotame, advantame, N-[N-[3-(3-hydroxy-4-methoxyphenyl)propyl]-L-α-aspartyl]-L-phenylalanine 1-methyl ester, N-(N-[3-(3-hydroxy-4-methoxyphenyl)-3-methylbutyl)-L-α-aspartyl]-L-phenylalanine 1-methyl ester, N-[N-[3-(3-methoxy-4-hydroxyphenyl)propyl]-L-α-aspartyl]-L-phenvlalanine 1-methyl ester, salts thereof and the like. Synthetic high-potency sweeteners also include modified synthetic high-potency sweeteners.

As used herein, and unless otherwise indicated, the term “sweetness intensity” is understood to mean any perceptible sweetness. For example, a comestible composition comprising at least one Compound provided herein may be slightly more sweet than a comestible composition without the at least one Compound. In at least one embodiment, a comestible composition provided herein is perceptibly more sweet than a comestible composition without the at least one Compound.

As used herein, and unless otherwise indicated, the term “trifluoromethyl” means —CF₃.

As used herein, and unless otherwise indicated, the phrase “undesirable taste” includes any taste property which is not imparted by sugars, e.g. glucose, sucrose, fructose, or similar saccharides. Non-limiting examples of undesirable tastes include delayed sweetness onset, lingering sweet aftertaste, metallic taste, bitter taste, cooling sensation taste or menthol-like taste, licorice-like taste, and/or the like.

As used herein, and unless otherwise indicated, the terms “zero-calorie” refers to a dietary sweetener composition that has a 100% or near 100% reduction in calories that would be associated with a full calorie sweetener composition.

It must be noted that, as used in the specification and the appended claims, the singular forms “a,” “an,” and “the” include plural referents unless the context clearly dictates otherwise. Thus, for example, reference to “an alkyl compound” includes mixtures of alkyl compounds.

It should be noted that if there is a discrepancy between a depicted structure and a name given that structure, the depicted structure is to be accorded more weight. Unless stated to the contrary, a formula with chemical bonds shown only as solid lines and not as wedges or dashed lines contemplates each possible isomer (e.g., each enantiomer and diastereomer, or geometric isomers (i.e., E, Z)), and a mixture of isomers, such as racemic or scalemic mixtures. Single stereochemical isomers, as well as enantiomeric and diastereomeric mixtures of a compound provided herein (such as a compound of Formula (I), Compound 1, Compound 2, Compound 3, Compound 4, Compound 5, Compound 6, Compound 7, Compound 8 or Compound 9) are within the scope of the invention. Further, unless otherwise stated, formulas depicted herein are also meant to include compounds which differ only in the presence of one or more isotopically enriched atoms. For example, compounds provided herein having the present formulas except for the replacement of a hydrogen by a deuterium or tritium, or the replacement of a carbon by a ¹³C— or ¹⁴C— enriched carbon are within the scope of tins invention.

A compound provided herein (such as a compound of Formula (I), Compound 1, Compound 2, Compound 3, Compound 4, Compound 5, Compound 6, Compound 7, Compound 8 or Compound 9) can also exist as solvates and hydrates. Thus, these compounds may crystallize with, for example, waters of hydration, or one, a number of, or any fraction thereof of molecules of the mother liquor solvent. The solvates and hydrates of such compounds are included within the scope of this invention.

### 3.2 Compounds

Provided herein are compounds of Formula (I):

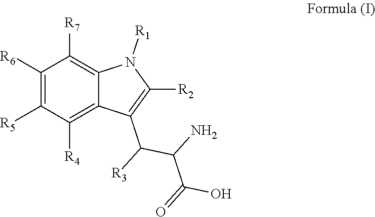

or a comestibly or biologically acceptable salt or stereoisomeric form thereof, wherein:
R₁ is hydrogen or C1-C6 alkyl;
R₂-R₁ and R₇ are each independently hydrogen, halogen, C1-C6 alkyl, C1-C₆ alkoxy, hydroxyl or trifluoromethyl; and
R₆ is I, hydrogen, C4-C6 alkyl, C4-C6 alkoxy or hydroxyl.

In certain embodiments, the compounds of Formula (I) provided herein are those wherein R₁, R₂, R₃, R₅, R₆, and R₇ are independently at each occurrence hydrogen.

In certain embodiments, the compounds of Formula (I) provided herein are those wherein R₄ is methyl.

In certain embodiments, the compounds of Formula (I) provided herein are those wherein the at least one compound is 2-amino-3-(4-methyl-1H-indol-3-yl)propanoic acid.

In certain embodiments, the compounds of Formula (I.) provided herein are those wherein R₁, R₂, R₃, R₅, R₆, and R₇ are independently at each occurrence hydrogen.

In certain embodiments, the compounds of Formula (I) provided herein are those wherein R₅ is methyl.

In certain embodiments, the compounds of Formula (I) provided herein are those wherein the at least one compound is 2-ammo-3-(5-methyl-1H-indol-3-yl)propanoic acid.

In certain embodiments, the compounds of Formula (I) provided herein are those wherein R₁, R₂, R₃, R₅, R₆, and R₇ are independently at each occurrence hydrogen.

In certain embodiments, the compounds of Formula (I) provided herein are those wherein R₂ is methyl.

In certain embodiments, the compounds of Formula (I) provided herein are those wherein the at least one compound is 2-amino-3-(2-methyl-1H-indol-3-yl)propanoic acid.

In certain embodiments, the compounds of Formula (I) provided herein are those wherein the compound is selected from the group consisting of the compounds in Table 1 or a comestibly or biologically acceptable salt or stereoisomeric form thereof.

TABLE 1
Compounds of Formula (I)

| Com- | | |
|---|---|---|
| pound | Compound Structure | Chemical Name* |
| 1 | 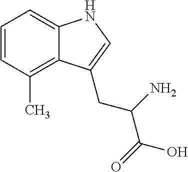 [SMILES: Cc1cccc2[nH]cc(CC(N)C(=O)O)c12] | 2-amino-3-(4-methyl- 1H-indol-3- yl)propanoic acid |
| 2 | 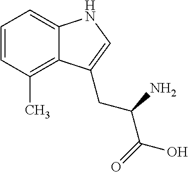 [SMILES: Cc1cccc2[nH]cc(C[C@@H](N)C(=O)O)c12] | (R)-2-amino-3-(4- methyl-1H-indol-3- yl)propanoic acid |
| 3 | 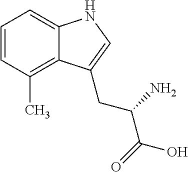 [SMILES: Cc1cccc2[nH]cc(C[C@H](N)C(=O)O)c12] | (S)-2-amino-3-(4- methyl-1H-indol-3- yl)propanoic acid |
| 4 | 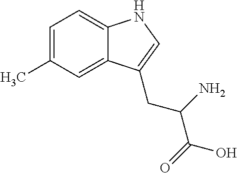 [SMILES: Cc1ccc2[nH]cc(CC(N)C(=O)O)c2c1] | 2-amino-3-(5-methyl- 1H-indol-3- yl)propanoic acid |
| 5 | 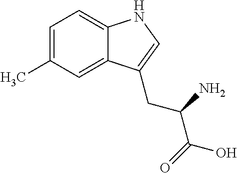 [SMILES: Cc1ccc2[nH]cc(C[C@@H](N)C(=O)O)c2c1] | (R)-2-amino-3-(5- methyl-1H-indol-3- yl)propanoic acid |
| 6 | 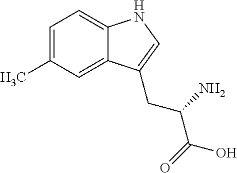 [SMILES: Cc1ccc2[nH]cc(C[C@H](N)C(=O)O)c2c1] | (S)-2-amino-3-(5- methyl-1H-indol-3- yl)propanoic acid |
| 7 | 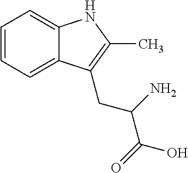 [SMILES: Cc1[nH]c2ccccc2c1CC(N)C(=O)O] | 2-amino-3-(2-methyl- 1H-indol-3- yl)propanoic acid |
| 8 | 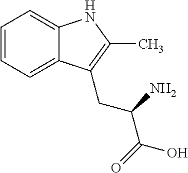 [SMILES: Cc1[nH]c2ccccc2c1C[C@@H](N)C(=O)O] | (R)-2-amino-3-(2- methyl-1H-indol-3- yl)propanoic acid |
| 9 | 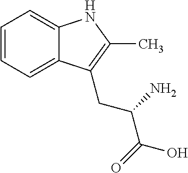 [SMILES: Cc1[nH]c2ccccc2c1C[C@H](N)C(=O)O] | (S)-2-amino-3-(2- methyl-1H-indol-3- yl)propanoic acid |

*Chemical Names automatically generated with ChemDraw Professional, Version 15.1.

For the purposes of this disclosure, Table 1 serves to define that a particular structure is associated with a particular name, Whenever a particular name is recited in this disclosure or the claims, the chemical structure associated with that particular name shall be the structure identified in Table 1.

In a particular embodiment, the compounds of Formula (I) provided herein are selected from the group consisting of:
2-amino-(-methyl-1H-indol-3-yl)propanoic acid (“Compound 1”);
(R)-2-amino-3-(4-methyl-1H-indol-3-yl)propanoic acid (“Compound 2”);
(S)-2-amino-3-(4-methyl-1H-indol-3-yl)propanoic acid (“Compound 3”);
2-amino-3-(5-methyl-1H-indol-3-yl)propanoic acid (“Compound 4”);
(R)-2-amino-3-(5-methyl-1H-indol-3-yl)propanoic acid (“Compound 5”);
(S)-2-amino-3-(5-methyl-1H-indol-3-yl)propanoic acid (“Compound 6”);
2-amino-3-(2-methyl-1H-indol-3-yl)propanoic acid (“Compound 7”);
(R)-2-amino-3-(2-methyl-1H-indol-3-yl)propanoic acid (“Compound 8”); and
(S)-2-amino-3-(2-methyl-1H-indol-3-yl)propanoic acid (“Compound 9”);
or a comestibly or biologically acceptable sail or stereoisomeric form thereof.

In certain embodiments, a compound provided herein (such as a compound of Formula (I), Compound 1, Compound 2, Compound 3, Compound 4, Compound 5, Compound 6, Compound 7, Compound 8 or Compound 9) is isolated, in certain embodiments, the compound provided herein is separated from its naturally occurring environment if it is derived from a natural source or product.

in certain embodiments, a compound provided herein (such as a compound of Formula (I), Compound 1, Compound 2, Compound 3, Compound 4, Compound 5, Compound 6, Compound 7, Compound 8 or Compound 9) is synthesized.

In certain embodiments, a compound provided herein (such as a compound of Formula (I), Compound 1, Compound 2, Compound 3, Compound 4, Compound 5, Compound 6, Compound 7, Compound 8 or Compound 9) is bio-synthesized.

In a particular embodiment, a compound provided herein (such as a compound of Formula (I), Compound 1, Compound 2, Compound 3, Compound 4, Compound 5, Compound 6, Compound 7, Compound 8 or Compound 9) is isolated and substantially pure. In certain embodiments, the compound provided herein is no less than about 95%, no less than about 96%, no less than about 97%, no less than about 98%, no less than about 98.5%, no less than about 99%, no less than about 99.5%, or no less than about 99.8% pure.

In a particular embodiment, a compound provided herein (such as a compound of Formula (I), Compound 1, Compound 4, or Compound 7) are those wherein the compound provided herein is racemic. In a particular embodiment, the compound provided herein is the L-isomer. In a more particular embodiment, the compound provided herein is the D-isomer.

In certain embodiments, the D-isomer of a compound provided herein (such as a D-isomer of a compound of Formula (I), Compound 2, Compound 5, or Compound 8) are those wherein the compound exhibits enhanced aqueous solubility over other isomers of the compound.

In certain embodiments, a compound provided herein (such as a compound of Formula (I), Compound 1, Compound 2, Compound 3, Compound 4, Compound 5, Compound 6, Compound 7, Compound 8 or Compound 9) modulates a sweet taste receptor in a subject. In certain embodiments, the activity of the compound provided herein to modulate a sweet taste receptor in a subject may be assessed using an in vitro assay that utilizes cells and cell lines that express or have been engineered to express one or more sweet taste receptors (see, e.g., WO/2014/176336 and WO/2013/059836 for exemplary assays that can be used, each of which is incorporated herein by reference in its entirety). In a particular embodiment, the sweet taste receptor is TAS1R2 or TAS1R3 or both of TAS1R2 and TAS1R3. In certain embodiments, the compound provided herein has an EC₅₀ value of at most 0.01 mM, 0.05 mM, 0.1 mM, 0.5 mM, 1 mM, 5 mM, 10 mM, 20 mM, or at most 30 mM in a cell-based sweet receptor assay, wherein the cell-based assay comprises contacting the at least one compound with a cell expressing TAS1R2+TAS1R3. In one embodiment, the cell-based assay further comprises measuring intracellular Ca²⁺ concentration using a Ca²⁺-sensitive fluorescent dye. In another embodiment, the EC₅₀ value is calculated by filling the data from the cell-based assay to the Hill's equation: Y=Bottom+(Top−Bottom)/(1+10 A((Log EC₅₀−X)*Hill Slope)), wherein X=log of dose or concentration, Y=Response (increasing as X increases), Top=maximum signal, and Bottom=minimum signal.

In certain embodiments, a compound provided herein (such as a compound of Formula (I), Compound 1, Compound 2, Compound 3, Compound 4, Compound 5, Compound 6, Compound 7, Compound 8 or Compound 9) may be present in an aqueous solution in an amount sufficient to impart a maximum sweetness intensity equivalent to that of a 20%

In certain embodiments, 50 mg-100 mg of a compound provided herein (such as a compound of Formula (I), Compound 1, Compound 2, Compound 3, Compound 4, Compound 5, Compound 6, Compound 7, Compound 8 or Compound 9) in 100 mg of water is equivalent to 1 degree Brix.

In certain embodiments, a compound provided herein (such as a compound of Formula (I), Compound 1, Compound 2, Compound 3, Compound 4, Compound 5, Compound 6, Compound 7, Compound 8 or Compound 9) provides from about 1 to about 12 degrees Brix when added to an unsweetened or sweetened beverage.

In certain embodiments, a compound provided herein (such as a compound of Formula (I), Compound 1, Compound 2, Compound 3, Compound 4, Compound 5, Compound 6, Compound 7, Compound 8 or Compound 9) imparts a more sugar-like characteristic to a comestible composition than a comestible composition without the compound. In a particular embodiment, between about 300 ppm to about 4000 ppm of the compound provided herein is sufficient to impart a desirable degree of a sugar-like characteristic to a comestible composition. In a particular embodiment, between about 150 ppm to about 300 ppm, about 300 ppm to about 4000 ppm of the compound provided herein is sufficient to impart a desirable degree of a sugar-like characteristic to a comestible composition. In certain embodiments, the sugar-like characteristic is selected from the group consisting of maximal response, flavor profile, temporal profile, adaptation behavior, mouthfeel, concentration/response function behavior, tastant and flavor/sweet taste interactions, and temperature effects.

In certain embodiments, a compound provided herein (such as a compound of Formula (I), Compound 1, Compound 2, Compound 3, Compound 4, Compound 5, Compound 6, Compound 7, Compound 8 or Compound 9) suppresses, reduces or eliminates at least one undesirable taste associated with sweeteners. In a particular embodiment, between about 150 ppm to about 4000 ppm of the compound provided herein is sufficient to suppress, reduce or eliminate at least one undesirable taste associated with sweeteners. In some embodiment the compound(s) provided herein is sufficient to suppress, reduce or eliminate at least one desirable taste associated with sweeteners when the compound(s) is present between about 150 ppm to about 200 ppm, about 200 ppm to about 300 ppm to about 400 ppm; about 400 ppm to about 500 ppm; about 500 ppm to about 600 ppm; about 600 ppm to about 700 ppm; about 700 ppm to about 800 ppm; about 800 ppm to about 900 ppm; about 900 ppm to about 1000 ppm; about 1000 ppm to about 1100 ppm; about 1100 ppm to about 1200 ppm; about 1200 ppm to about 1300 ppm; about 1300 ppm to about 1400 ppm; about 1400 ppm to about 1500 ppm; about 1500 ppm to about 1600 ppm; about 1600 ppm to about 1700 ppm; about 1700 ppm to about 1800 ppm; about 1800 ppm to about 1900 ppm; about 1900 ppm to about 2000 ppm; about 2000 ppm to about 2100 ppm; about 2100 ppm to about 2200 ppm; about 2200 ppm to about 2300 ppm; about 2300 ppm to about 2400 ppm; about 2400 ppm to about 2500 ppm; about 2500 ppm to about 2600 ppm; about 2600 ppm to about 2700 ppm; about 2700 ppm to about 2800 ppm, about 2800 ppm to about 2900 ppm, about 2900 ppm to about 3000 ppm, about 3000 ppm to about 3100 ppm about 3100 ppm to about 3200 ppm, about 3200 ppm to about 3300 ppm, about 3300 ppm to about 3400 ppm, about 3400 ppm to about 3500 ppm about 3500 ppm to about 3600 ppm, about 3600 ppm to about 3700 ppm, about 3700 ppm to about 3800 ppm about 3800 ppm to about 3900, or about 3900 to about 4000 ppm or increments in between those recited. In a particular embodiment, the at least one undesirable taste is selected from the group consisting of delayed sweetness onset, lingering sweet aftertaste, metallic taste, bitter taste, cooling sensation taste, menthol-like taste, or licorice-like taste.

A comestibly or biologically acceptable salt of a compound provided herein (such as a compound of Formula (I), Compound 1, Compound 2, Compound 3, Compound 4, Compound 5, Compound 6, Compound 7, Compound 8 or Compound 9) includes salts preferably derived from inorganic or organic acids and bases. Examples of such salts include, but are not limited to, those derived from appropriate bases, including alkali metal (e.g., sodium and potassium), alkaline earth metal (e.g., magnesium), ammonium and N⁺(C₁₋₄ alkyl)₄ salts. In a particular embodiment, the comestibly or biologically acceptable salt of the compound provided herein is a sodium salt, a potassium salt, or a calcium salt. In a more particular embodiment, the comestibly or biologically acceptable salt of the compound provided herein is a sodium salt.

In certain embodiments, the sodium salt of a compound provided herein (such as a compound of Formula (I), Compound 1, Compound 2, Compound 3, Compound 4, Compound 5, Compound 6, Compound 7, Compound 8 or Compound 9) is capable of disintegrating or dissolving 300-400 times more rapidly than the :free base of the compound.

### 3.3 Methods of Making

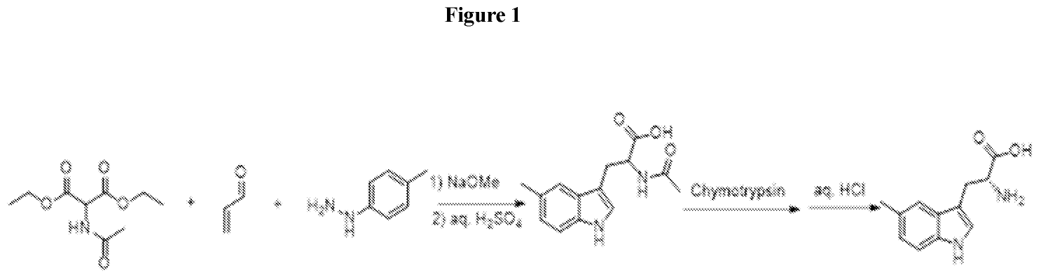

A compound provided herein (such as a compound of Formula (I), Compound 1, Compound 2, Compound 3, Compound 4, Compound 5, Compound 6, Compound 7, Compound 8 or Compound 9) may be synthesized using conventional methods known to those of ordinary skill in the art and commercially available materials. For example, particular compounds provided herein can be prepared as outlined in FIG. 1. It should be noted that one skilled in the art can modify the procedures set forth in the illustrative schemes and examples to arrive at the desired product. D-5-Methyl tryptophan was prepared as showed in Fig. 1 and includes Nucleophilic addition of diethyl acetamidomaionate to acrolein by sodium methoxide, followed by the addition of phenylhydrazine to form the malonate phenylhydrazone, and cyclization of phenylhydrazone with aq. sulfuric acid yielded racemic acetyl D-5-methyl tryptophan, Enzymatic hydrolysis of racemic acetyl 5-methyl tryptophan with □-chymotrypsin produced unhydrolyzed D-isomer, which was extracted with organic solvents and then hydrolyzed with aq. HCl to give the desired D-5-methyl tryptophan (Synthesis, resolution and characterization of ring substituted phenylalanines and tryptophans by John Porter, John Dykert and Jean Rivier, Int. J. Peptide Protein Res. 1987, 30, 13-21).

Comestible Compositions

In certain embodiments, provided herein is a comestible composition comprising at least one compound provided herein (such as a compound of Formula (I), Compound 1, Compound 2, Compound 3, Compound 4, Compound 5, Compound 6, Compound 7, Compound 8 or Compound 9) or a comestibly or biologically acceptable salt thereof, as described in Section 5.2, which incorporated herein by reference in its entirety. In certain embodiments, the at least one compound provided herein is 2-amino-3-(4-methyl-1H-indol-3-yl)propanoic acid. In certain embodiments, the at least one compound provided herein is 2-amino3-(5-methyl-1H-indol-3-yl)propanoic acid. In certain embodiments the at least one compound provided herein is 2-amino-3-(2-methyl-1H-indol-3-yl)propanoic acid.

In certain embodiments, the comestible composition provided herein is a dietary sweetener composition, a consumable, a food or beverage mix, or a pharmaceutical composition.

In a particular embodiment, the comestible composition provided herein comprises at most 0.1%, 0.5%, 1%, 2.5%, 5%. 7.5%, 10%, 25%, 50%, 75%, 90%, or at most 95% by weight of a compound provided herein (such as a compound of Formula (I), Compound 1, Compound 2, Compound 3, Compound 4, Compound 5, Compound 6, Compound 7, Compound 8 or Compound 9).

In a particular embodiment, the comestible composition provided herein comprises at least 0.1%, 0.5%, 1%, 2.5%, 5%, 7.5%, 10%, 25%, 50%, 75%, 90%, or at least 95% by weight of a compound provided herein (such as a compound of Formula (I), Compound 1, Compound 2, Compound 3, Compound 4, Compound 5, Compound 6, Compound 7, Compound 8 or Compound 9).

In certain embodiments, the comestible composition provided herein is enclosed in a package tor storing and dispensing the comestible composition. Comestible compositions are embodied and packaged in numerous different forms and it is intended that the comestible compositions provided herein may be of any form known in the art. In accordance with particular embodiments, non-limiting examples of comestible compositions include powder form, granular form, packets, tablets, sachets, pellets, cubes, solids, and liquids.

### (a) Dietary Sweetener Compositions

In certain embodiments, the comestible composition provided herein is a dietary sweetener composition. In certain embodiments, the dietary sweetener composition provided herein comprises at least one compound provided herein (such as a compound of Formula (I), Compound 1, Compound 2, Compound 3, Compound 4, Compound 5, Compound 6, Compound 7, Compound 8 or Compound 9) or a comestibly or biologically acceptable salt thereof, as described in Section 5.2, which incorporated herein by reference in its entirety. In certain embodiments, the at least one compound provided herein is 2-amino-3-(4-methyl-1H-indol-3-yl)propanoic acid. In certain embodiments, the at least one compound provided herein is 2-amino-3-(5-methyl-1H-indol-3-yl)propanoic acid. In certain embodiments, the at least one compound provided herein is 2-amino-3-(2-methyl-1H-indol-3-yl)propanoic acid.

In certain embodiments, the dietary sweetener composition provided herein comprises about 0.03 to about 0.4 % of a compound provided herein (such as a compound of Formula (I), Compound 1, Compound 2, Compound 3, Compound 4, Compound 5, Compound 6, Compound 7, Compound 8 or Compound 9) by weight of the total composition.

In certain embodiments, the dietary sweetener composition provided herein comprises at least one compound provided herein (such as a compound of Formula (I), Compound 1, Compound 2, Compound 3, Compound 4, Compound 5, Compound 6, Compound 7, Compound 8 or Compound 9) and at least one additive. In certain embodiments, the combination of the at least one compound provided herein and the at least one additive enhances the sugar-like characteristics, including the temporal profile and/or flavor profile of the composition, such as the osmotic taste, of the dietary sweetener composition. In certain embodiments, the combination of the at least one compound provided herein and the at least one additive results in a lower total amount of the at least one additive that is required to achieve the same level of sweetness associated with the individual additive. One of ordinary skill in the art, with the teachings of the present invention, may arrive at all the possible combinations of the at least one compound provided herein (such as a compound of Formula (I), Compound 1, Compound 2, Compound 3, Compound 4, Compound 5, Compound 6, Compound 7, Compound 8 or Compound 9) and the at least one additive.

In a particular embodiment, the additive is selected from the group consisting of carbohydrates, polyols, amino acids and their corresponding salts, poly-amino acids and their corresponding salts, sugar acids and their corresponding salts, nucleotides, organic acids, inorganic acids, organic salts including organic acid salts and organic base salts, inorganic salts, bitter compounds, flavorants and flavoring ingredients, astringent compounds, proteins or protein hydrolysates, surfactants, emulsifiers, weighing agents, gums, antioxidants, colorants, flavonoids, alcohols, polymers and combinations thereof.

Suitable carbohydrate additives include, but arc not limited to, carbohydrate additives with a molecular weight ranging from about 50 to about 500. Non-limiting examples of carbohydrate additives with a molecular weight ranging from about 50 to about 500 include sucrose, fructose, glucose, maltose, lactose, mannose, galactose, ribose, rhamnose, trehalose, and tagatose.

Suitable polyol additives include, but arc not limited to, polyol additives with a molecular weight ranging from about 76 to about 500. Non-limiting examples of polyol additives with a molecular weight ranging from about 76 to about 500 include erythritol, glycerol, and propylene glycol. In other embodiments, other suitable polyol additives include sugar alcohols.

Suitable amino acid additives for use in embodiments of this disclosure include, but are not limited to, aspartic acid, arginine, glycine, glutamic acid, proline, threonine, theanine, cysteine, cystine, alanine, valine, tyrosine, leucine, isoleucine, asparagine, serine, lysine, histidine, ornithine, methionine, carnitine, aminobutyric acid (α-, β-, or γ-isomers), glutamine, hydroxyproline, taurine, norvaline, sarcosine, and their salt forms such as sodium or potassium salts or acid salts. The salty taste improving amino acid additives also may be in the D- or L-configuration and in the mono-, di-, or tri-form of the same or different amino acids. Additionally, the amino acids may be α-, β-, γ-, δ-, and ε-isomers if appropriate. Combinations of the foregoing amino acids and their corresponding salts (e.g., sodium, potassium, calcium, magnesium salts or other alkali or alkaline earth metal salts thereof, or acid salts) also are suitable additives in some embodiments. The amino acids may be natural or synthetic. The amino acids also may be modified. Modified amino acids refers to any amino acid wherein at least one atom has been added, removed, substituted, or combinations thereof (e.g., N-alkyl amino acid, N-acyl amino acid, or N-methyl amino acid). Non-limiting examples of modified amino acids include amino acid derivatives such as trimethyl glycine, N-methyl-glycine, and N-methyl-alanine. As used herein, modified amino acids encompass both modified and unmodified amino acids. As used herein, amino acids also encompass both peptides and polypeptides (e.g., dipeptides, tripeptides, tetrapeptides, and pentapeptides) such as glutathione and L-alanyl-L-glutamine. Suitable polyamino acid additives include poly-L-aspartic acid, poly-L-lysine (e.g., poly-L-α-lysine or poly-L-ε-lysine), poly-L-ornithine (e.g., poly-L-α-ornithine or poly-L-ε-ornithine), poly-L-arginine, other polymeric forms of amino acids, and salt forms thereof (e.g., calcium, potassium, sodium, or magnesium salts such as L-glutamic acid mono sodium salt), lire poly-amino acid additives also may be in the D- or L-configuration. Additionally, the poly-amino acids may be α-, β-, γ-, δ-, and ε-isomers if appropriate. Combinations of the foregoing poly-amino acids and their corresponding salts (e.g., sodium, potassium, calcium, magnesium salts or other alkali or alkaline earth metal salts thereof or acid salts) also are suitable additives in some embodiments. The poly-amino acids described herein also may comprise co-polymers of different amino acids. The poly-amino acids may be natural or synthetic. The poly-amino acids also may be modified, such that at least one atom has been added, removed, substituted, or combinations thereof (e.g., N-alkyl poly-amino acid or N-acyl poly-amino acid). As used herein, poly-amino acids encompass both modified and unmodified poly-amino acids. For example, modified poly-amino acids include, but are not limited to poly-amino acids of various molecular weights (MW), such as poly-L-α-lysine with a MW of 1,500, MW of 6,000, MW of 25,200, MW of 63,000, MW of 83,000, or MW of 300,000. In some embodiments, the amino acid additive is glycine, alanine, taurine, serine or proline. In such embodiments, the amino acid additive is present in a concentration of about 10 ppm to about 25,000 ppm or about 100 ppm to about 1000 ppm.

Suitable inorganic acid salt additives include, but are not limited to, inorganic acid salt additives with a molecular weight from about 58 to about 120. Non-limiting examples of inorganic acid salt additives with a molecular weight from about 58 to about 120 include sodium chloride, potassium chloride, magnesium chloride, magnesium phosphate, NaHSO₄.H₂O, KH₂PO₄, NaH₂PO₄, and KAl(SO₄)₂ and combinations thereof.

Suitable bitter compounds include, but are not limited to, caffeine, quinine, urea, quassia, tannic acid, bitter orange oil, naringin, and salts thereof.

Suitable protein or protein hydrolysate additives include, but are not limited to, protein or protein hydrolysate additives with a molecular weight ranging from about 75 to about 300. Non-limiting examples of protein or protein hydrolysate additives with a molecular weight ranging from about 75 to about 300 include protein or protein hydrolysates containing glycine, alanine, serine, leucine, valine, isolcucine, proline, hydroxyproline, glutamine, and threonine.

Suitable alcohol additives include, but are not limited to, alcohol additives with a molecular weight ranging from about 46 to about 500. A non-limiting example of sweet taste improving alcohol additive with a molecular weight ranging from about 46 to about 500 includes ethanol.

In a particular embodiment, the additive is a sweetener. In certain embodiments, the sweetener is selected from the group consisting of sucrose, fructose, glucose, high fructose corn syrup, xylose, arabinose, rhamnose, erythritol, xylitol, mannitol, sorbitol, inositol, AceK, aspartame, neotame, sucralose, saccharine, naringin dihydrochalcone (NarDHC), neohesperidin dihydrochalcone (NDHC), rubusoside, stevia, rebaudioside A, stevioside, mogroside IV, siamenoside I, mogroside V, trilobatin, rebaudioside A, rebaudioside B, rebaudioside C (dulcoside B), rebaudioside D, rebaudioside E, rebaudioside F, rebaudioside I, rebaudioside H, rebaudioside L, rebaudioside K, rebaudioside J, rebaudioside N, rebaudioside O, rebaudioside M, dulcoside A, rebaudioside X, glycosylated steviol glycosides, mogrosides, isomogroside, Luo Han Guo fruit extract, monatin and its salts (monatin SS, RR, RS, SR), curculin, glycyrrhizic acid and its salts, thaumatin, monellin, mabinlin, brazzein, hernandulcin, phyllodulcin, glycyphyllin, phloridzin, baiyunoside, osladin, polypodoside A, pterocaryoside A, pterocaryoside B, mukurozioside, phlornisoside I, periandrin I, abrusoside A, and cyclocariosidc I.

In certain embodiments, the dietary sweetener composition provided herein is a zero-, low-, or mid-caloric sweetener composition, optionally containing caffeine. Those of ordinary skill in the art should appreciate that the dietary sweetener composition can be customized to obtain a desired calorie content. For example, a low-caloric or non-caloric dietary sweetener composition and/or other caloric additives may be combined with a caloric natural sweetener to produce a sweetener composition with a preferred calorie content.

In certain embodiments, the dietary sweetener composition provided herein is a tabletop sweetener composition. In certain embodiments, the tabletop sweetener composition provided herein comprises at least one compound provided herein (such as a compound of Formula (I), Compound 1, Compound 2, Compound 3, Compound 4, Compound 5, Compound 6, Compound 7, Compound 8 or Compound 9), at least one additive, and optionally, at least one bulking agent. In certain embodiments, the bulking agent is selected from the group consisting of maltodextrin (e.g., 10 DE, 18 DE, or 5 DE), com syrup solids (e.g., 20 or 36 DE), sucrose, fructose, glucose, invert sugar, sorbitol, xylose, ribulose, mannose, xylitol, mannitol, galactitol, erythritol, maltitol, lactitol, isomalt, maltose, tagatose, lactose, inulin, glycerol, propylene glycol, polyols, polydextrose, fructooligosaccharides, cellulose and cellulose derivatives, or combinations thereof. Additionally, granulated sugar (sucrose) or other caloric sweeteners such as crystalline fructose, other carbohydrates, or sugar alcohols can be used as a bulking agent due to their provision of good content uniformity without the addition of significant calories.

Tabletop sweetener compositions provided herein are embodied and packaged in numerous different forms and it is intended that die tabletop sweetener compositions provided herein may be of any form known in the art. Non-limiting examples of forms of the tabletop sweetener composition provided herein include powder, granular, packets, tablets, sachets, pellets, cubes, solids, liquids, suspensions, syrups, and emulsions.

In certain embodiments, the tabletop sweetener composition provided herein comprises a single-serving (portion control) packet comprising a dry-blend of a dietary sweetener formulation. Such dry-blend formulations generally may comprise powder or granules. Although the dietary sweetener formulation may be in a packet of any size. A non-limiting example of conventional portion control tabletop packets are approximately 2.5 by 1.5 inches and hold approximately 1 gram of a dietary sweetener formulation having a sweetness equivalent to 2 teaspoons of granulated sugar (approximately 8 g). In certain embodiments, the conventional portion control tabletop package holds no more than 1 gram of dietary sweetener formulation. In other embodiments, the conventional portion control tabletop package hold no less than 1 gram of dietary sweetener formulation. The amount of a compound provided herein (such as a compound of Formula (I), Compound 1, Compound 2, Compound 3, Compound 4, Compound 5, Compound 6, Compound 7, Compound 8 or Compound 9) in a dry-blend tabletop dietary sweetener formulation will vary due to the varying potency of the compounds. In a particular embodiment, a dry-blend tabletop dietary sweetener formulation may comprise a compound provided herein (such as a compound of Formula (I), Compound 1, Compound 2, Compound 3, Compound 4, Compound 5, Compound 6, Compound 7, Compound 8 or Compound 9) in an amount from about 1% (w/w) to about 10% (w/w) of the tabletop dietary sweetener composition. The single-serving tabletop sweetener compositions provided herein may be flavored or unflavored.

In certain embodiments, the tabletop sweetener composition provided herein may exist in the form of a solid. In a particular embodiment, a solid tabletop sweetener composition includes cubes and tablets. A non-limiting example of conventional cubes are equivalent in size to a standard cube of granulated sugar, which is approximately 2.2×2.2×2.2 cm³ and weigh approximately 8 g. In one embodiment, a solid tabletop sweetener composition is in the form of a tablet or any other form known to those skilled in the art.

In certain embodiments, the tabletop sweetener composition provided herein may exist in the form of a liquid, wherein a compound provided herein (such as a compound of Formula (I), Compound 1, Compound 2, Compound 3, Compound 4, Compound 5, Compound 6, Compound 7, Compound 8 or Compound 9) is combined with a liquid carrier. Non-limiting examples of carrier agents for liquid tabletop sweetener compositions include water, alcohol, polyol, glycerin base or citric acid base dissolved in water, and mixtures thereof. Due to the varying potencies of the different high-potency sweeteners, the amount of high-potency sweetener in a liquid tabletop sweetener composition will also vary. The sweetness equivalent of a tabletop sweetener composition provided herein for any of the forms described herein or known in the an may be varied to obtain a desired sweetness profile. For example, in one embodiment, a tabletop sweetener composition provided herein may comprise a sweetness comparable to that of an equivalent amount of standard sugar. In another embodiment, the tabletop sweetener composition provided herein may comprise a sweetness of up to 100 times that of an equivalent amount of sugar. In another embodiment, the tabletop sweetener composition provided herein may comprise a sweetness of up to 90 times, 80 times, 70 times, 60 times, 50 times, 40 times, 30 times, 20 times, 10 times, 9 times, 8 times, 7 times, 6 times, 5 times, 4 times, 3 times, and 2 times that of an equivalent amount of sugar.

In certain embodiments, the tabletop sweetener composition provided herein also may be formulated for targeted uses, for example, in beverage, food, pharmaceutical, cosmetics, herbal/vitamins, tobacco, and in any other products which may be sweetened. For example, a tabletop sweetener composition provided herein for baking may be formulated having additional protecting agents, such as encapsulants. Other forms will be readily apparent to those skilled in the tabletop sweetener art.

Commonly used methods for making powder or granulated tabletop sweetener formulations tor packets include fluid bed agglomeration processes. These processes typically involve spraying finely divided particles of a solution onto a fluidized bed of particles under moisture and temperature conditions which promote formation of an agglomerate. The solution comprises a compound provided herein (such as a compound of Formula (I), Compound 1, Compound 2, Compound 3. Compound 4, Compound 5, Compound 6, Compound 7, Compound 8 or Compound 9), an additive, and a binding agent. The spray rate can be modified to control the average particle size. It is known that by increasing the spray rate, the average particle size is also increased, following the spraying of the particles, the particles are allowed to dry and may optionally be screened to control the particle size distribution.

In another embodiment for making a powder or granulated tabletop sweetener composition for packets, the method comprises combining at least one compound provided herein (such as a compound of Formula (I), Compound 1, Compound 2, Compound 3, Compound 4, Compound 5, Compound 6, Compound 7, Compound 8 or Compound 9) and any additive or bulking agent with an aqueous solution to form an aqueous suspension that is thoroughly blended. The suspension is then heated to approximately 50° C. to 90° C. under vacuum to remove the water while avoiding decomposition of the materials. Finally, the mixture is milled to the desired particle size.

Those skilled in the art appreciate that the amount of a compound provided herein (such as a compound of Formula (I), Compound 1, Compound 2, Compound 3, Compound 4, Compound 5. Compound 6, Compound 7, Compound 8 or Compound 9) and the amount and types of additives and/or bulking agents can be modified in order to tailor the taste of the tabletop sweetener composition to a desired profile and end use. It is contemplated that other methods for making tabletop sweetener compositions that are well known in the art also may be used.

### (b) Consumable

In certain embodiments, the comestible composition provided herein is a consumable. In certain embodiments, the consumable provided herein comprises at least one compound provided herein (such as a compound of Formula (I), Compound 1, Compound 2, Compound 3, Compound 4, Compound 5, Compound 6, Compound 7, Compound 8 or Compound 9) or a comestibly or biologically acceptable salt thereof, as described in Section 3.2, which incorporated herein by reference in its entirety. In certain embodiments, the at least one compound provided herein is 2-amino-3-(4-methyl-1H-indol-3-yl)propanoic acid. In certain embodiments, the at least one compound provided herein is 2-amino-3-(5-methyl-1H-indol-3-yl)propanoic acid. In certain embodiments, the at least one compound provided herein is 2-amino-3-(2-methyl-1H-indol-3-yl)propanoic acid.

In certain embodiments, the consumable provided herein comprises about 0.03% to 0.4% of a compound provided herein (such as a compound of Formula (I), Compound 1, Compound 2, Compound 3, Compound 4, Compound 5, Compound 6, Compound 7, Compound 8 or Compound 9) by weight of the total composition.

In a particular embodiment, the consumable provided herein is a beverage or a food. In a more particular embodiment, the consumable provided herein is a beverage. In certain embodiments, the beverage is a zero-, low-, and mid-calorie beverage, optionally containing caffeine. Non-limiting examples of beverages include non-carbonated beverages, carbonated beverages, fruit-flavored beverages, citrus-flavored beverages, root beer, fruit juices, fruit-containing beverages, vegetable juices, vegetable-containing beverages, teas, coffees, sports drinks, energy drinks, milk, nutritional drinks in the form of shakes, malts, and the like, and flavored waters.

### (c) Food or Beverage Mixes

In certain embodiments, the comestible composition provided herein is a food or beverage mix. In certain embodiments, the food or beverage mix provided herein comprises at least one compound provided herein (such as a compound of Formula (I), Compound 1, Compound 2, Compound 3, Compound 4. Compound 5, Compound 6, Compound 7, Compound 8 or Compound 9) or a comestibly or biologically acceptable salt thereof, as described in Section 5.2, which incorporated herein by reference in its entirety. In certain embodiments, the at least one compound provided herein is 2-amino-3-(4-methyl-1H-indol-3-yl)propanoic acid. In certain embodiments, the at least one compound provided herein is 2-amino-3-(5-methyl-1H-indol-3-yl)propanoic acid. In certain embodiments, the at least one compound provided herein is 2-amino-3-(2-methyl-1H-indol-3-yl)propanoic acid.

In certain embodiments, the food or beverage mix provided herein comprises about 0.03% to 0.4% of a compound provided herein (such as a compound of Formula (I), Compound 1, Compound 2, Compound 3, Compound 4, Compound 5, Compound 6, Compound 7, Compound 8 or Compound 9) by weight of the total composition.

In a particular embodiment, the comestible composition provided herein is a beverage mix. In a more particular embodiment, the beverage mix provided herein is a zero-, low-, and mid-calorie beverage mix, optionally containing caffeine. Non-limiting examples of beverage mixes provided herein include sugar-free beverages, soft drinks, fortified beverages, milk, hot chocolate, instant iced teas, instant coffees, nutritional drinks in the form of shakes, malts, and the like, nutritional supplements, and fruit-flavored beverages.

In a particular embodiment, the comestible composition provided herein is a food mix. In a more particular embodiment, the food mix provided herein is a zero-, low-, and mid-calorie food mix. Non-limiting examples of food mixes provided herein include gelatin dessert, pudding, sauce, gravy, soup, dressing, mousse, vegetable dip, frozen dessert, whipped topping, and coffee creamer.

In some embodiments, the food or beverage mix provided herein is a dry mix. Food or beverage mixes provided herein are embodied and packaged in numerous different forms and it is intended that the food or beverage mixes provided herein may be of any form known in the art. Non-limiting examples of forms of the food or beverage mixes provided herein include powder, granular, packets, tablets, sachets, pellets, cubes, solids, liquids, suspensions, syrups, and emulsions.

In certain embodiments, the food or beverage mix provided herein comprises an additive as described in Section 5.4(a). which is incorporated herein by reference in its entirety. In certain embodiments, the food or beverage mix provided herein comprises other additives, such as natural or synthetic aromas or flavors/flavoring agents (e.g., lemon, orange, grapefruit, strawberry, banana, pear, kiwi, grape, apple, lemon, mango, pineapple, passion fruit, raspberry, jaxnaica, marigold, chrysanthemum, tea, chamomile, ginger, valerian, yohimbe, hops, eriodictyon, ginseng, bilberry, rice, red wine, mango, peony, lemon balm, nut gall, oak chip, lavender, walnut, gentiarn, luo han guo, cinnamon, angelica, aloe, agrimony, yarrow, and mixtures thereof), coloring agents (e.g., FD&C dyes such as yellow #5, blue ∩2, red #40 and/or FD&C lakes), additional sweeteners, preservatives, vitamins (e.g., iron, zinc, vitamin C, calcium, vitamin A, vitamin C, niacin, thiamin, vitamin B6, vitamin B2, vitamin B12, folic acid, and iodine), minerals, thickening agents (e.g., arboxymethylcellulose (CMC), carrageenan, xanthan, pectin, guar and various food starches (modified and unmodified), com syrup solids and vegetable oils or partially hydrogenated vegetable oils), antioxidants (e.g., butylated hydroxyanisole (BBA), butylated hydroxytoluene (BHT), and mixtures thereof), and the like.

In certain embodiments, the food or beverage mix provided herein comprises at least one compound provided herein (such as a compound of Formula (I), Compound 1, Compound 2, Compound 3, Compound 4, Compound 5, Compound 6, Compound 7, Compound 8 or Compound 9) and a water soluble flow agent. Non-limiting examples of a water soluble flow agent include sodium carboxymethyl cellulose, dextran, algin, gum arabic, carrageenan, xanthan gum, guar gum, hydroxy-propylmethyl cellulose (HPMC), methyl cellulose, pectin, locust bean gum, sodium alginate, propylene glycol alginate, caramel and mixtures thereof. Trace amount of an emulsifier or wetting agent such as polysorbate (polyoxyethylene fatty acid ester) or lecithin may also be incorporated to improve the dissolution and stability characteristics of the food or beverage mix.

In certain embodiments, the food or beverage mix provided herein further comprises at least one encapsulation agent. Non-limiting examples of an encapsulation agent include carbohydrates such as the dextrins (e.g., malodextrin), gum arable, and starches (e.g., hydrolyzed starch such as Sta-Mist 515 and Mira-Cap, modified starches such as N-Lok and Capsul, modified potato starch such as Amylogum CLS).

In certain embodiments, the food or beverage mix provided herein may be prepared by a variety of means such as dry blending the ingredients, spray drying, agglomeration, drum drying and other conventional means of providing a dry mix of uniform consistency. For example, the food or beverage mix provided herein may be prepared by incorporation of a small amount of a water soluble flow agent into an aqueous solution of a compound provided herein (such as a compound of Formula (1;, Compound 1, Compound 2, Compound 3, Compound 4, Compound 5, Compound 6, Compound 7, Compound 8 or Compound 9) to create a suspension. The suspension of the compound provided herein and the water soluble flow agent is then combined with an aqueous solution of an encapsulation agent(s) and mixed thoroughly. The combined suspension is then dried (e.g., spray drying) to form a powdered or dry mix. Those skilled in the art appreciate that the amount of a compound provided herein (such as a compound of Formula (I), Compound 1, Compound 2, Compound 3, Compound 4, Compound 5, Compound 6, Compound 7, Compound 8 or Compound 9) and the amount and types of water soluble flow agent and encapsulation agent(s) can be modified in order to tailor the suspension to a desired profile and end use. It is contemplated that other methods for making food or beverage mixes that are well known in the art also may be used.

### (d) Pharmaceutical Compositions

In certain embodiments, the comestible composition provided herein is a pharmaceutical composition. In certain embodiments, the pharmaceutical composition provided herein comprises at least one compound provided herein (such as a compound of Formula (I), Compound 1, Compound 2, Compound 3, Compound 4, Compound 5, Compound 6, Compound 7, Compound 8 or Compound 9) or a comestibly or biologically acceptable salt thereof, as described in Section 5.2, which incorporated herein by reference in its entirety. In certain embodiments, the at least one compound provided herein is 2-amino-3-(4-methyl-1H-indol-3-yl)propanoic acid. In certain embodiments, the at least one compound provided herein is 2-amino-3-(5-methyl-1H-indol-3-yl)propanoic acid. In certain embodiments, the at least one compound provided herein is 2-amino-3-(2-methyl-1H-indol-3-yl)propanoic acid.

In certain embodiments, the comestibly or biologically acceptable salt of a compound provided herein (such as a compound of Formula (I), Compound 1, Compound 2, Compound 3, Compound 4, Compound 5, Compound 6, Compound 7, Compound 8 or Compound 9) is also a pharmaceutically acceptable salt.

In certain embodiments, the pharmaceutical composition provided herein comprises about 0.03% to 0.4% of a compound provided herein (such as a compound of Formula (.1), Compound 1, Compound 2, Compound 3, Compound 4, Compound 5, Compound 6, Compound 7, Compound 8 or Compound 9) by weight of the total composition.

In a particular embodiment, the pharmaceutical composition provided herein further comprises an active pharmaceutical ingredient and an excipient.

In a particular embodiment, the pharmaceutical composition provided herein is formulated for oral administration, buccal administration, sublingual administration, or any other route of administration as known in the art. In oral, buccal, or sublingual administration embodiments of pharmaceutical compositions, the pharmaceutical composition provided herein can mask a bitter or otherwise undesirable taste of an active pharmaceutical ingredient or an excipient.

Non-limiting examples of suitable active pharmaceutical ingredients include, but are not limited to, antacids, reflux suppressants, antiflatulents, antidopaminergics, proton pump inhibitors, cytoprotectants, prostaglandin analogues, laxatives, antispasmodics, antidiarrhoeals, bile acid sequestrants, opioids, beta-receptor blockers, calcium channel blockers, diuretics, cardiac glycosides, antiarrhythmics, nitrates, antianginals, vasoconstrictors, vasodilators, peripheral activators, ACE inhibitors, angiotensin receptor blockers, alpha blockers, anticoagulants, heparin, antiplatelet drugs, fibrinolytics, anti-hemophilic factors, haemostatic drugs, hypolipidaemic agents, statins, hynoptics, anaesthetics, antipsychotics, antidepressants, anti-emetics, anticonvulsants, antiepileptics, anxiolytics, barbiturates, movement disorder drugs, stimulants, benzodiazepines, cyclopyrrolones, dopamine antagonists, antihistamines, cholinergics, anticholinergics, emetics, cannabinoids, analgesics, muscle relaxants, antibiotics, aminoglycosides, anti-virals, anti-fungals, anti-inflammatories, anti-gluacoma drugs, sympathomimetics, steroids, ceruminolytics, bronchodilators, NSAIDS, antitussive, mucolytics, decongestants, corticosteroids, androgens, antiandrogens, gonadotropins, growth hormones, insulin, antidiabetics, thyroid hormones, calcitonin, diphosponates, vasopressin analogues, alkalizing agents, quinolones, anticholinesterase, sildenafil, oral contraceptives, Hormone Replacement Therapies, bone regulators, follicle stimulating hormones, luteinizings hormones, gamolenic acid, progestogen, dopamine agonist, oestrogen, prostaglandin, gonadorelin, clomiphene, tamoxifen, diethylstilbestrol, antileprotics, amituberculous drugs, antimalarials, anthelmintics, antiprotozoal, antiserums, vaccines, interferons, tonics, vitamins, eydotoxic drugs, sex hormones, aromatase inhibitors somatostatin inhibitors, or similar type substances, or combinations thereof.

Non-limiting examples of suitable excipients include but are not limited to, antiadherents, binders microcrystalline cellulose, gum tragacanth, or gelatin), coatings, disintegrants, fillers, diluents, softeners, emulsifiers, flavoring agents coloring agents, adjuvats, lubricants, functional agents (e.g., nutrients), viscosity modifiers, bulking agents, glidiants (e.g., colloidal silicon dioxide) surface active agents, osmotic agents, diluents or any other non-active ingredient, or combinations thereof. For example, the pharmaceutical compositions provided herein may include excipient materials selected from tile group consisting of calcium carbonate, coloring agents, whiteners, preservatives, flavors, triacetin, magnesium stearate, sterotes natural or artificial flavors essential oils, plant extracts, fruit essences, gelatins, or combinations thereof.

In certain embodiments, the pharmaceutical composition provided herein may be in the form of a tablet, a capsule, a liquid, an aerosol, a powder, an effervescent tablet or powder, a syrup, an emulsion, a suspension, a solution or any other form for providing the pharmaceutical composition to a subject.

The pharmaceutical compositions provided herein may be prepared using known techniques. Generally, pharmaceutical compositions may be manufactured by acquiring the active pharmaceutical ingredient can by chemical synthesis, extraction, cell culture or fermentation, recovery from natural sources, or a combination of these processes. The active pharmaceutical ingredient can then be physically processed by tableting, preparing capsules, preparing solutions, or other pharmaceutical preparation methods which properly dose the active pharmaceutical ingredient. For example, in tableting, a compound provided herein (such as a compound of Formula (I), Compound 1Compound 2, Compound 3, Compound 4, Compound 5, Compound 6, Compound 7, Compound 8 or Compound 9), an active pharmaceutical ingredient, and an excipient should be dry, powdered, and of uniform grain size as possible. Mixed grain sizes tend to separate out due to operational vibrations, resulting in inconsistent tableting, while any moisture in the system will tend to clog the tableting pathways. Binders, disintegrants, lubricants, and/or coatings may also be used as excipient in the tablet to be formed from the pharmaceutical composition. The dry ingredients are then pressed into a tablet having the proper dose of the active pharmaceutical ingredient.

In another embodiment, a compound provided herein (such as a compound of Formula (I), Compound 1, Compound 2, Compound 3, Compound 4, Compound 5, Compound 6, Compound 7, Compound 8 or Compound 9), an active pharmaceutical ingredient, and an excipient, are combined to form a solution of a pharmaceutical composition. In some embodiments, the solution could comprise a solvent and a propellant and be used as an aerosol. In other embodiments, the solution could comprise a syrup and be orally introduced into a subject.

### 3.4 Methods of Use

### (a) Methods of Preparing a Comestible Composition

Provided herein are methods of preparing a comestible composition, comprising (a) providing a comestibly acceptable carrier; and (b) adding to the comestibly acceptable carrier at least one compound provided herein (such as a compound of Formula (I), Compound 1, Compound 2, Compound 3. Compound 4, Compound 5, Compound 6, Compound 7, Compound 8 or Compound 9) or a comestibly or biologically acceptable salt thereof, as described in Section 5.2, which incorporated herein by reference in its entirety. In certain embodiments, the at least one compound provided herein is 2-amino-3-(4-methyl-1H-indol-3-yl)propanoic acid. In certain embodiments, the at least one compound provided herein is 2-amino-3-(5-methyl-1H-indol-3-yl)propanoic acid. In certain embodiments, the at least one compound provided herein is 2-amino-3-(2-methyl-1H-indol-3-yl)propanoic acid.

In certain embodiments, the comestible composition provided herein is a dietary sweetener composition, a consumable, a food or beverage mix, or a pharmaceutical composition. In a preferred embodiment, the comestible composition is a consumable. In a more preferred embodiment, the consumable is a beverage.

In a particular embodiment, the comestible composition provided herein comprises at most 0.1%, 0.5%, 1%, 2.5%, 5%, 7.5%, 10%, 25%, 50%, 75%, 90%, or at most 95% by weight of a compound provided herein (such as a compound of Formula (I), Compound 1, Compound 2, Compound 3, Compound 4, Compound 5, Compound 6, Compound 7, Compound 8 or Compound 9).

In a particular embodiment, the comestible composition provided herein comprises at least 0.1%, 0.5%, 1%, 2.5%, 5%, 7.5%, 10%, 25%, 50%, 75%, 90%, or at least 95% by weight of a compound provided herein (such as a compound of Formula (I), Compound 1, Compound 2, Compound 3, Compound 4, Compound 5, Compound 6, Compound 7, Compound 8 or Compound 9).

The comestibly acceptable carrier may be a solid or a liquid. Generally, non-limiting examples of comestibly acceptable carriers include many common food ingredients, such as water at neutral, acidic, or basic pH, fruit or vegetable juices, vinegar, marinades, beer, wine, natural water/fat emulsions such as milk or condensed milk, edible oils and shortenings, fatty acids, low molecular weight oligomers of propylene glycol, glyceryl esters of fatty acids, and dispersions or emulsions of such hydrophobic substances in aqueous media, salts such as sodium chloride or potassium chloride, wheat flours, solvents such as ethanol, solid edible diluents such as vegetable powders or flours, or other liquid vehicles, dispersion or suspension aids, surface active agents, thickening or emulsifying agents, preservatives, binding agents, lubricants, and the like. Non-limiting examples of comestibly acceptable liquid carriers include water, ethanol, propylene glycol, glycerin, triacetin, edible fats or oils comestibly acceptable glyceride triesters, benzyl alcohol, triethyl citrate, and benzyl benzoate. Non-limiting examples of comestibly acceptable solid carriers include edible polysaccharides such as natural or modified starches, vegetable flours, maltodextrin, polyphosphate, alginate, chitosan, carrageenan, pectin, starch, gum arabic, alfa-lactalbumin, beta-lactoglobumin, ovalbumin, polysorbitol, cyclodextrin, cellulose, methyl cellulose, ethyl cellulose, hydropropylmethylcellulose, carboxymethylcellulose, powdered milk, milk protein, whey protein, soy protein, canola protein, albumin, and gelatin.

### (b) Methods of Enhancing Sweetness

Provided herein are methods for enhancing the sweetness of a consumable, comprising (a) providing a consumable; and (b) adding to the consumable a comestible composition comprising at least one compound provided herein (such as a compound of Formula (I), Compound 1, Compound 2, Compound 3, Compound 4, Compound 5, Compound 6, Compound 7, Compound 8 or Compound 9) or a comestibly or biologically acceptable salt thereof, as described in Section 5.2, which incorporated herein by reference in its entirety. In certain embodiments, the at least one compound provided herein is 2-amino-3-(4-methyl-1H-indol-3-yl)propanoic acid. In certain embodiments, the at least one compound provided herein is 2-amino-3-(5-methyl-1H-indol-3-yl)propanoic acid. In certain embodiments, the at least one compound provided herein is 2-amino-3-(2-methyl-1H-indol-3-yl)propanoic acid.

In a particular embodiment, the comestible composition provided herein comprises at most 0.1%, 0.5%, 1%, 2.5%, 5%, 7.5%, 10%, 25%, 50%, 75%, 90%, or at most 95% by weight of a compound provided herein (such as a compound of Formula (I), Compound 1, Compound 2, Compound 3, Compound 4, Compound 5, Compound 6, Compound 7, Compound 8 or Compound 9).

In a particular embodiment, the comestible composition provided herein comprises at least 0.1%, 0.5%, 1%, 2.5%, 5%, 7.5%, 10%, 25%, 50%, 75%, 90%, or at least 95% by weight of a compound provided herein (such as a compound of Formula (I), Compound 1, Compound 2, Compound 3, Compound 4, Compound 5, Compound 6, Compound 7, Compound 8 or Compound 9).

In certain embodiments, a compound provided herein (such as a compound of Formula (I), Compound 1, Compound 2, Compound 3, Compound 4, Compound 5, Compound 6, Compound 7, Compound 8 or Compound 9) enhances the sweetness of the consumable by an amount sufficient to impart a maximum sweetness intensity equivalent to that of a 20% aqueous solution of sucrose by weight.

Non-limiting examples of suitable consumables include, but are not limited to, liquid-based or dry consumables, such as, for example, pharmaceutical compositions, edible gel mixes and compositions, dental compositions, foodstuffs, beverages, and beverage products. In a preferred embodiment, the consumable is a beverage.

In certain embodiments, a compound provided herein (such as a compound of Formula (I),

Compound 1, Compound 2, Compound 3, Compound 4, Compound 5, Compound 6, Compound 7, Compound 8, Compound 9, or combinations of the foregoing compounds) increases sweetness in a manner not solely attributable to the inherent sweetness of the compounds) as provided herein alone. Generally, the compounds provided herein (which may serve to enhance the perception of sweetness) may enhance or potentiate the sweet taste of sweeteners without providing any noticeable sweet taste by themselves at or below a sweetness threshold level; however, the compound(s) as provided herein may themselves provide sweet taste at concentrations above a sweetness threshold level. It is noted that the compound(s) as provided herein, may be effective as enhancers even if present at concentrations above their sweetness threshold level. In such embodiments, there is major contribution of the compound(s) (such as a compound of Formula (I), Compound 1, Compound 2, Compound 3, Compound 4, Compound 5, Compound 6, Compound 7, Compound 8, Compound 9, or combinations of the foregoing compounds) to the sweetness of the composition via enhancement of the inherently sweet taste attributed to a sweetener, where the sweetener is also present in the composition. As used herein, the term “sweetness threshold ” and “sweetness recognition threshold”, are used interchangeably herein, and refer to the level at which the lowest known concentration of a certain sweet compound is perceivable by the human sense of taste. The sweetness threshold level varies for different edible compositions (e.g., in different matrices), and may be varied with respect to the individual perceiving the sweetness.

In certain embodiments of this disclosure, when the compound(s) as disclosed herein are used above their sweetness threshold level, they synergize with sweeteners to enhance or potentiate the perception of sweetness due to the sweetener. In such cases, the overall sweetness of a composition comprising the compound(s) as disclosed herein (such as a compound of Formula (I), Compound 1, Compound 2, Compound 3, Compound 4, Compound 5, Compound 6, Compound 7, Compound 8, Compound 9, or combinations of the foregoing compounds) and a sweetener is higher than the combined inherent sweetness due to the sweetener and the compound(s) as disclosed herein. Such an increase in perceived sweetness may be referred to as synergistic or super-additive, not additive.

In certain embodiments enhancement of sweetness is measured by comparing the perception of sweetness of a composition comprising the compound(s) to a control composition not comprising the compound(s). In certain embodiments of the present disclosure, the compound(s) provided herein (such as a compound of Formula (I), Compound 1, Compound 2, Compound 3, Compound 4, Compound 5, Compound 6, Compound 7, Compound 8, Compound 9, or combinations of the foregoing compounds) is a different compound from the sweetener in the composition, in certain embodiments the compound(s) as provided herein (such as a compound of Formula (I), Compound 1, Compound 2, Compound 3, Compound 4. Compound 5, Compound 6, Compound 7, Compound 8, Compound 9, or combinations of the foregoing compounds) is the sweetener in the composition. In certain embodiments of the present disclosure, the sweetener and the sweetness enhancer in the composition is a compound(s) as disclosed herein (such as a compound of Formula (I), Compound 1, Compound 2, Compound 3, Compound 4, Compound 5, Compound 6, Compound 7, Compound 8, Compound 9, or combinations of the foregoing compounds), but the sweetener compound(s) is different from the sweetness enhancing compound.

In some embodiments the compound(s) as disclosed herein (such as a compound of Formula (I), Compound 1, Compound 2, Compound 3, Compound 4, Compound 5, Compound 6, Compound 7, Compound 8, Compound 9, or combinations of the foregoing compounds) do not by themselves provide sweetness (are not inherently sweet) when the compound(s) is present at concentration between about 10 ppm to about 150 ppm.

In some embodiments the compound(s) as disclosed herein (such as a compound of Formula (I), Compound 1, Compound 2, Compound 3, Compound 4, Compound 5, Compound 6, Compound 7, Compound 8, Compound 9, or combinations of the foregoing compounds) do not by themselves provide sweetness (are not inherently sweet) when the compound(s) is present at concentration between about 10 ppm to about 20 ppm; between about 20 ppm to about 30 ppm; between about 30 ppm to about 40 ppm; between about 40 ppm to about 50 ppm; between about 50 ppm to about 60 ppm; between about 60 ppm to about 70 ppm; between about 70 ppm to about 80 ppm; between about 80 ppm to about 90 ppm; between about 90 ppm to about 100 pm; between about 100 ppm to about 110 ppm; between about 110 ppm to about 120 ppm; between about 120 ppm to about 130 ppm; between about 130 ppm to about 140 ppm; between about 140 ppm to about 150 ppm, or increments in between those recited.

In some embodiments the compound(s) as disclosed herein (such as a compound of Formula (I), Compound 1, Compound 2, Compound 3, Compound 4, Compound 5, Compound 6, Compound 7, Compound 8, Compound 9, or combinations of the foregoing compounds) do not by themselves provide sweetness (are not inherently sweet) when the compound(s) is present at concentration of no more than 10 ppm, 20 ppm, 30 ppm, 40 ppm, 50 ppm, 60 ppm, 70 ppm, 80 ppm, 90 ppm, 100 ppm, 110 ppm, 120 ppm, 130 ppm, 140 ppm, 150 ppm. or increments in between those recited.

### (c) Methods of Enhancing Sugar-Like Characteristics

Provided herein are methods for enhancing the sugar-like characteristics of a consumable, comprising (a) providing a consumable; and (b) adding to the consumable a comestible composition comprising at least one compound provided herein (such as a compound of Formula (I), Compound 1, Compound 2, Compound 3, Compound 4, Compound 5, Compound 6, Compound 7. Compound 8 or Compound 9) or a comestibly or biologically acceptable salt thereof, as described in Section 5.2, which incorporated herein by reference in its entirety. In certain embodiments, the at least one compound provided herein is 2-amino-3-(4-methyl-1H-indol-3-yl)propanoic acid. In certain embodiments, the at least one compound provided herein is 2-amino-3-(5-methyl-1H-indol-3-yl)propanoic acid. In certain embodiments, the at least one compound provided herein is 2-amino-3-(2-methyl-1H-indol-3-yl)propanoic acid.

In a particular embodiment, the comestible composition provided herein comprises at most 0.1%, 0.5%, 1%, 2.5%, 5%, 7.5%, 10%, 25%, 50%, 75%, 90%, or at most 95% by weight of a compound provided herein (such as a compound of Formula (I), Compound 1, Compound 2, Compound 3, Compound 4, Compound 5, Compound 6, Compound 7, Compound 8 or Compound 9).

In a particular embodiment, the comestible composition provided herein comprises at least 0.1%, 0.5%, 1%, 2.5%, 5%, 7.5%, 10%, 25%, 50%, 75%, 90%, or at least 95% by weight of a compound provided herein (such as a compound of Formula (I), Compound 1, Compound 2, Compound 3, Compound 4, Compound 5, Compound 6, Compound 7, Compound 8 or Compound 9).
In certain embodiments, a compound provided herein (such as a compound of Formula (I), Compound 1, Compound 2, Compound 3, Compound 4, Compound 5, Compound 6, Compound 7, Compound 8 or Compound 9) enhances the sugar-like characteristic of the comestible composition in comparison to a comestible composition without the compound. In a particular embodiment, between about 150 ppm to about 4000 ppm of the compound provided herein is sufficient to impart a desirable degree of a sugar-like characteristic to a comestible composition. In a particular embodiment between about 300 ppm to about 4000 ppm of the compound provided herein is sufficient to impart a desirable degree of a sugar-like characteristic to a comestible composition. In certain embodiments the compound(s) provided herein impart a desirable degree of a Sugar-like characteristic to a comestible composition when the compound(s) is present at a concentration between about 150 ppm to 200 ppm, about 200 ppm to about 300 ppm to about 400 ppm; about 400 ppm to about 500 ppm, about 500pm to about 600 ppm, about 600 ppm to about 700 ppm; about 700 ppm to about 800 pppm; about 800 ppm to about 900 ppm; about 900 ppm to about 1000 ppm; about 1000 ppm to about 1100 ppm; about 1100 ppm to about 1200 ppm; about 1200 ppm to about 1300 ppm; about 1300 ppm to about 1400 ppm; about 1400 ppm to about 1500 ppm; about 1500 ppm to about 1600 ppm; about 1600 ppm to about 1700 ppm; about 1700 ppm to about 1800 ppm; about 1800 ppm to about 1900 ppm; about 1900 ppm to about 2000 ppm: about 2000 ppm to about 2100 ppm; about 2100 ppm to about 2200 ppm; about 2200 ppm to about 2300 ppm; about 2300 ppm to about 2400 ppm; about 2400 ppm to about 2500 ppm; about 2500 ppm to about 2600 ppm; about 2600 ppm to about 2700 ppm; about 2700 ppm to about 2800 ppm; about 2800 ppm to about 2900 ppm, or about 2900 ppm to about 3000 ppm or increments in between those recited.
In certain embodiments the compound(s) provided herein impart a desirable degree of a sugar-like characteristic to a comestible composition when the compound(s) is present at a concentration of at least 150 ppm, 200 ppm, 300 ppm, 400 ppm, 500 ppm, 600 ppm, 700 ppm, 800 ppm, 900 ppm, 1000 ppm, 1100 ppm, 1200 ppm, 1300 ppm, 1400 ppm, 1500 ppm, 1600 ppm, 1700 ppm, 1800 ppm, 1900 ppm, 2000 ppm, 2100 ppm, 2200 ppm, 2300 ppm, 2400 ppm, 2500 ppm, 2600 ppm, 2700 ppm, 2800 ppm, 2900 ppm, 3000 ppm or increments in between those recited.
   In certain embodiments, the sugar-like characteristic is selected from the group consisting of maximal response, flavor profile, temporal profile, adaptation behavior, mouthfeel, concentration/response function behavior, tastant and flavor/sweet taste interactions, and temperature effects.

Non-limiting examples of suitable consumables include, but are not limited to, liquid-based or dry consumables, such as, for example, pharmaceutical compositions, edible gel mixes and compositions, dental compositions, foodstuffs, beverages, and beverage products. In a preferred embodiment, the consumable is a beverage.

### 4. EXAMPLES

In order that this invention be more fully understood, the following examples are set forth. These examples are only for the purpose of illustration and are not to be construed as limiting the scope of the invention in any way.

The test compounds used in the following examples may be obtained from commercial vendors. Compound 1 (cat.no.AK394270) and 8 (cat.no. AK688785) can be obtained from Ark Pharm. Compound 5 can be obtained from Amatek Chemical, (cat.no. A-0797) and from Ark Pharm Inc. (Product AK167448). D-Tryptophan can be obtained from Sigma-Aldrich (Product T9753) and AK Scientific (Product A644).

### Example 1

### Descriptive Analysis Sensory Evaluation

### Effect of Test Compounds on the Perception of Sweetness in Humans Using a Trained Descriptive Analysis (DA) Panel

The effect of the test compound on the perception of sweet taste in an aqueous matrix was evaluated using a descriptive analysis methodology with a group of trained panelists, as follows.

Preparation of Samples for Sensory Taste Tests:
Control Sample: 500 ppm Rebaudioside M
Variant Sample: 1500 ppm Compound 5
   Samples for the descriptive analysis taste test were prepared by dissolving, both the control and variant samples, directly in water at the desired test concentrations.

Sensory Methodology:

The taste test panelists used in Example I were selected using a sensory acuity screening program and then trained in descriptive analysis taste testing. Candidate panelists were recruited, with prescreening and personal interviews, and were assessed for their ability to detect, recognize and differentiate basic taste attributes or mixtures thereof as part of a standardized acuity test. These candidate panelists were also assessed for their innate ability to identify flavors, and to rank on intensity scales. Other senses such as smell and vision were also included as part of the assessment. Candidates also were screened for their ability to use the language to describe and articulate ideas.

The method was performed as a double blinded, randomized test, where trained Descriptive Analysis taste panelists (n≥12) profiled the maximum intensity perceived at 18 seconds, of the control sample and the variant sample for the following 3 attributes; sweet, bitter and sweet appearance time. In addition to the maximum intensity, the panelists continued to assess sweet and bitter intensity (at 1 min, 1.5 min, 2 min, and 3 min), to generate a discontinuous time scale Intensity profile (TSI) of sweet and bitter percepts to gain an understanding of the taste profile of the control and variant samples. The results of such assessment are set forth in Table 1. Each panelist scored sweetness intensity on a 15 cm scale, with the control and variant sample being determined to be iso-sweet by ANOVA at 18 seconds. Both for sweet and bitter, the lingering effects were significantly less by ANOVA in the Compound sample, at multiple time points, as compared to Die Rebaudioside M sample. Thereby demonstrating that the taste profile was improved by Compound 5 as compared to the Rebaudiosidc M sample.

TABLE 1
DA assessment for sweet and bitter attributes for Compound 5

| | 500 ppm | 1500 ppm | |
|---|---|---|---|
| Descriptive | Rebaudioside | Compound | Significantly |
| Analysis | M | 5 | Different? |
| Parameter | (control) | (variant) | (by ANOVA) |
| Sweet @ 18 sec | 10.7 | 10.8 | NO (isosweet) |
| Sweet @ 1 min | 7.2 | 6.5 | YES (sweet linger |
| | | | improvement |
| Sweet @ | 5.3 | 4.6 | YES (sweet linger |
| 1 min 30 s | | | improvement |
| Sweet @ 2 min | 3.6 | 3 | NO (lower intensity) |
| Bitter @ 18 sec | 3.3 | 2.2 | YES (bitter |
| | | | improvement) |
| Bitter @ 1 min | 2.2 | 1.2 | YES (bitter linger |
| | | | improvement) |
| Bitter @ | 1.4 | 0.5 | YES (bitter linger |
| 1 min 30 s | | | improvement) |
| Bitter @2 min | 0.7 | 0.1 | YES (bitter linger |
| | | | improvement) |
| Bitter @3 min | 0.3 | 0.1 | YES (bitter linger |
| | | | improvement) |
| Sweet AT | 5.9 | 4.8 | YES (sweet AT |
| | | | improvement) |

### Example 2

### Discriminative Sensory Evaluation ps Evaluation of Sweetness Intensity of Test Compounds in Humans Using Discrimination Sensory Testing

The test compounds were evaluated for sweetness intensity in a aqueous solution in humans using a two-alternative forced choice “sip and spit” method (2AFC).

Preparation of Samples for Sensory Taste Tests:
Control Sample: Sucrose (various levels)
Variant Sample: Compound 1, 5 and 8

The control samples for the discrimination taste test were prepared by dissolving the sucrose directly in water at the indicated test concentrations Variant sample containing Compound 1 was prepared by dissolving Compound i directly in water at the desired concentration. Variant sample containing Compound 5 and 8 were prepared by dissolving the respective sodium salts of the compounds directly in water (refer to Example 4).

### Sensory Methodology:

The 2AFC test used for compound evaluation was a double blinded and randomized test where taste panelists (n≥12) evaluate a pair of sweetened solutions at a time—one sample is sweetened with sucrose (i.e. control) while the other sample is sweetened with the test compound (i.e. variant). Panelists were instructed not to eat or drink (except water) for at least 1 h before the test. During the test, panelists were instructed to sip each sample, swirl it around their mouth and then expectorate. After tasting each sample in the pair, panelists were instructed to record the sample that is “sweeter” in taste. Panelists cleansed their palates by rinsing with water, eating a cracker and waiting for an interval of about 5 minutes. All samples were tasted at ambient temperatures. Data were analyzed using binomial probabilities. The results of the 2AFC analysis are presented in Table 2, below

TABLE 2
Discrimination Data Summary via 2AFC in water

| | | | | Number | |
|---|---|---|---|---|---|
| Compound | Variant | Control | Total | picking | p |
| No. | Sample | Sample | Number | Variant | value |
| 1 | Compound | 10% | 38 | 25 | 0.036 |
| | 1 @ | Sucrose | | | |
| | 1000 ppm | | | | |
| 5 | Compound | 12% | 38 | 26 | 0.017 |
| | 5 @ | Sucrose | | | |
| | 2000 ppm | | | | |
| 8 | Compound | 8% | 20 | 11 | 0.412 |
| | 8 @ | Sucrose | | | |
| | 3000 ppm | | | | |

### Example 3

### Evaluation of Compound 5 and Compound 8 in a Mammalian Cell System Expressing a Sweet Taste Receptor

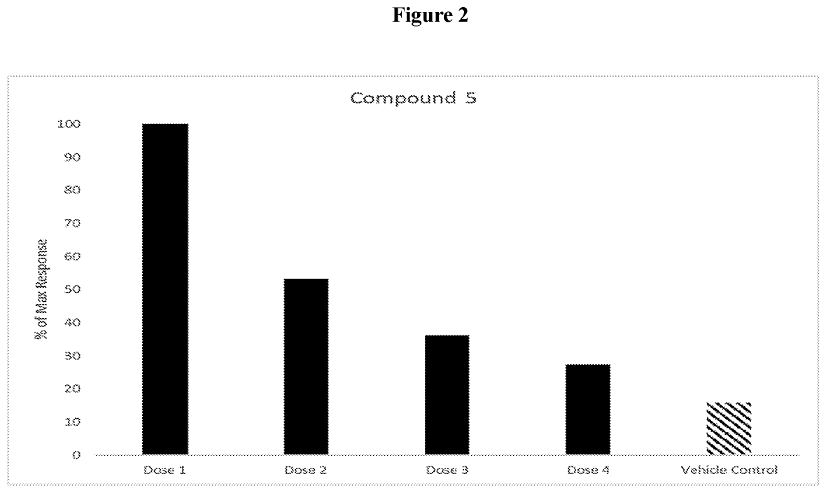

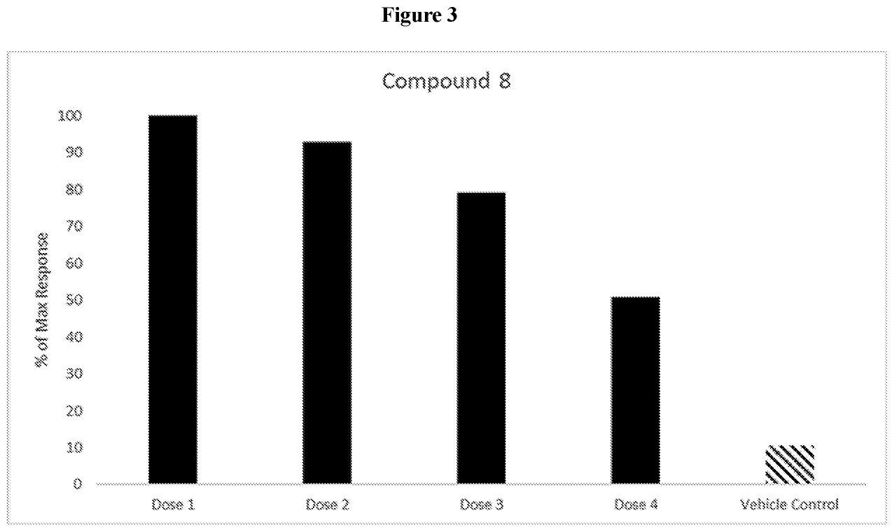

Compound 5 and Compound 8 were evaluated for their effects in a mammalian cell system expressing a sweet taste receptor (T1R2/T1R3). Compounds 5 and 8 were prepared in a standard assay buffer solution consisting of (in mM): 5.4 KCl, 1.8 CaCl₂, 12.5 NaHCO3, 0.4 MgSO₄, 12.5 HEPES, 1.0 Na₂HPO₄, 5.5 Glucose, pH 7.4, 116 NaCl;. A concentration response curve for Compounds 5 and 8 was prepared in assay buffer in appropriate concentrations to achieve the final on-cell concentrations of in mM: 15.3, 10.2, 6.8, 4.5, 3.0, 2.0, 1.3, 0.9. Cells were incubated with a calcium responsive dye for one hour at 37 C and then exposed to Compound 5 and 8 as well as vehicle control. Change in fluorescence prior to compound addition and after compound addition was monitored for up to six minutes using a Hamamatsu Functional Drug Screening System (FDSS) 6000. Data were analyzed by evaluating the signal over background for each individualized concentration of Compound 5. FIGS. 2 and 3 outlines the activity evaluated for the given compounds evaluated by this method.

### Example 4

### Solubility

Compounds 5 and 8 have been prepared at an increased concentration for commercial applications by converting them into their respective sodium salt forms. The process of converting to a salt form was obtained by dissolving the Compounds in water and adding a set quantity of sodium hydroxide or potassium hydroxide solution. A concentrated solution of 30% by weight of Compound 5 and 8 has been prepared by this method. This has been achieved by combining 30%, by weight, of Compound 5 or Compound 8 with 5.5%, by weight, of sodium hydroxide in de-ionized water. The mixture is subjected to intermittent vortex and sonication for a duration of 30-120 s. This process achieves a 300 times concentrated stock compared to the free base form of Compound 5 and 8, thereby enabling delivery of an increased concentration of the compounds in a model beverage/matrix. The sodium/potassium salt can increase solubility by changing neutral zwitterion to more hydrophilic negative charged anion.

### Example 5

### Sweetness Potency Comparison

The sweetness potency of the test compound was compared D-Tryptophan to understand potency differences between the test compound and non-methylated variants of D-tryptophan.

Preparation of Samples for Taste Tests:
Control Sample: 250 ppm, 500 ppm and 1000 ppm of D-Tryptophan
Variant Sample: 250 ppm, 500 ppm and 1000 ppm of Compound 5
   Samples for the taste test were prepared by dissolving, both the control and variant samples, directly in water at the desired test concentrations.

Sensory Methodology:

Expert tasters (n>5) tasted the control and test samples in a blinded experiment and assessed sweetness intensity (at the same concentration) via paired comparison. Each taster evaluated a pair of sweetened solutions at a time—one sample is sweetened with Control compound (i.e. control) while the other sample is sweetened with the test compound (i.e. variant). After tasting each sample in the pair, tasters were instructed to record the sample that is “sweeter” in taste. All samples were tasted at ambient temperatures. The results of such assessment are set forth in Table 3. At each tested dose, Compound 5 was picked “sweeter” compared to Control sample in the paired comparison, by all tasters demonstrating that Compound 5 offers higher potency, in comparison to Control compound, when tested, at the same concentration.

TABLE 3
Paired Comparison of Control vs Compound 5

| | | % picking Test as |
|---|---|---|
| Test Sample | Control Sample | “sweeter” |
| Compound 5 @ 250 ppm | Control @ 250 ppm | 100% |
| Compound 5 @ 500 ppm | Control @ 500 ppm | 100% |
| Compound 5 @ 1000 ppm | Control @ 1000 ppm | 100% |

In another test, expert tasters (n>5) also tasted Compound 5 and Control Samples to a set of sucrose references (5%, 7.5%, 10%) and ranked the Test and Control samples for sweetness intensity compared to sucrose references. The results for this experiment are set forth in Table 4. At the same concentration, Compound 5 achieves higher (close to 2 fold) potency compared to Control compound.

TABLE 4
Reference Scaling of Control vs Compound 5

| Sample | Sweetness Intensity (avg) |
|---|---|
| Compound 5 @ 1000 ppm | 9.25% Sucrose equivalent |
| Control @ 1000 ppm | 5.83% Sucrose equivalent |

The results show that methylation at select positions unexpectedly increases the sweetness potency of D-tryptophan.

1-61. (canceled)

62. A comestible composition comprising at least one compound of Formula (I): 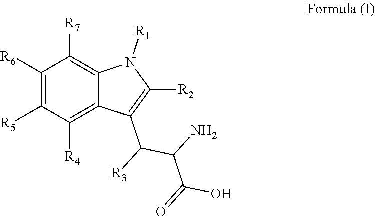
or a comestibly or biologically acceptable salt or stereoisomeric form thereof, wherein:
R₁is hydrogen or C1-C6 alkyl;
R₂-R₅ and R₇ are each independently hydrogen, halogen, C1-C6 alkyl, C1-C6 alkoxy, hydroxyl or trifluoromethyl; and
R₆ is I, hydrogen, C4-C6 alkyl, C4-C6 alkoxy or hydroxyl.

63. The comestible composition of claim 62, wherein R₁, R_{2,} R_{3,} R_{4,} R_{6,} and R₇ are independently at each occurrence hydrogen.

64. The comestible composition of claim 63, wherein R₅ is methyl.

65. The comestible composition of claim 62, wherein the at least one compound is 2-amino-3-(5-methyl-1H-indol-3-yl)propanoic acid.

66. The comestible composition of claim 62, wherein the comestible composition comprises a racemic mixture of the at least one compound.

67. The comestible composition of claim 62, wherein the at least one compound is the D-isomer.

68. The comestible composition of claim 62, wherein the at least one compound is the L-isomer.

69. The comestible composition of claim 62, wherein the comestibly or biologically acceptable salt is selected from the group consisting of a sodium salt, a potassium salt, or a calcium salt.

70. A dietary sweetener composition comprising the comestible composition of claim 62.

71. The dietary sweetener composition of claim 70 further comprising at least one additive in addition to the compound of Formula (I).

72. The dietary sweetener composition of claim 71, wherein the additive is selected from the group consisting of carbohydrates, polyols, amino acids and their corresponding salts, poly-amino acids and their corresponding salts, sugar acids and their corresponding salts, nucleotides, organic acids, inorganic acids, organic salts including organic acid salts and organic base salts, inorganic salts, bitter compounds, flavorants and flavoring ingredients, astringent compounds, proteins or protein hydrolysates, surfactants, emulsifiers, weighing agents, gums, antioxidants, colorants, flavonoids, alcohols, polymers and combinations thereof.

73. The dietary sweetener composition of claim 70, wherein the sweetener composition is a tabletop sweetener composition.

74. The dietary sweetener composition of claim 70, wherein the sweetener composition is a zero-, low-, or mid-calorie sweetener composition, optionally containing caffeine.

75. A consumable comprising the comestible composition or the dietary sweetener composition of claim 70.

76. The consumable of claim 75, wherein the consumable is a beverage or a food.

77. The consumable of claim 76, wherein the consumable is a beverage.

78. The consumable of claim 77, wherein the beverage is selected from a zero-, low-, and mid-calorie beverage, optionally containing caffeine.

79. A method of preparing a comestible composition, comprising:
a providing a comestibly acceptable carrier; and
b. adding to the comestibly acceptable carrier at least one compound of Formula (I) 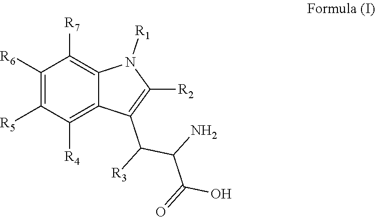
or a comestibly or biologically acceptable salt or stereoisomeric form thereof, wherein:
R₁ is hydrogen or C1-C6 alkyl;
R₂-R₅ and R₇ are each independently hydrogen, halogen, C1-C6 alkyl, C1-C6 alkoxy, hydroxyl or trifluoromethyl; and
R₆ is I, hydrogen, C₄-C₆ alkyl, C₄-C₆ alkoxy or hydroxyl.

80. The method of claim 79, wherein the at least one compound is 2-amino-3-(5-methyl-1H-indol-3-yl)propanoic acid.

81. The comestible composition of claim 67, wherein the D-isomer of the at least one compound has the following structure: 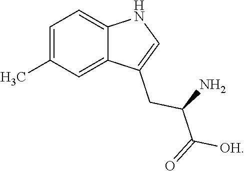 [SMILES: Cc1ccc2[nH]cc(C[C@@H](N)C(=O)O)c2c1]
